SEQUENCE LISTING (43 sequences)

SEQ ID NO: 1 - DNA, 119 bp, synthetic construct
  source 1..119: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 2 - DNA, 63 bp, synthetic construct
  source 1..63: mol_type = other DNA
ccctagaaagataatcatattgtgacgtacgttaaagataatcatgcgtaaaattgacgcatg

SEQ ID NO: 3 - DNA, 175 bp, synthetic construct
  source 1..175: mol_type = other DNA
tgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 4 - DNA, 231 bp, synthetic construct
  source 1..231: mol_type = other DNA
agggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 5 - DNA, 218 bp, Pongo abelii
  source 1..218: mol_type = genomic DNA
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 6 - DNA, 100 bp, Mus musculus
  source 1..100: mol_type = genomic DNA
cactgggaggatgttgagtaagatggaaaactactgatgacccttgcagagacagagtattaggacatgtttgaacaggggccgggcgatcagcaggtag

SEQ ID NO: 7 - DNA, 202 bp, Mus musculus
  source 1..202: mol_type = genomic DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 8 - DNA, 82 bp, synthetic construct
  source 1..82: mol_type = other DNA
gtaagggtttaagggatggttggttggtggggtattaatgtttaattacctggagcacctgcctgaaatcactttttttcag

SEQ ID NO: 9 - DNA, 1386 bp, synthetic construct
  source 1..1386: mol_type = other DNA
atgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtga

SEQ ID NO: 10 - DNA, 278 bp, Homo sapiens
  source 1..278: mol_type = genomic DNA
ctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 11 - DNA, 192 bp, synthetic construct
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 12 - DNA, 231 bp, synthetic construct
  source 1..231: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 13 - DNA, 274 bp, synthetic construct
  source 1..274: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgtcatttagaaagagagagcaatatttcaagaatg

SEQ ID NO: 14 - DNA, 35 bp, unidentified
  source 1..35: mol_type = genomic DNA
catgcgtcaattttacgcagactatctttctaggg

SEQ ID NO: 15 - DNA, 894 bp, synthetic construct
  source 1..894: mol_type = other DNA
gaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcg

SEQ ID NO: 16 - DNA, 161 bp, synthetic construct
  source 1..161: mol_type = other DNA
cctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 134 bp, synthetic construct
  source 1..134: mol_type = other DNA
acacagatccacaagctcctgctagcaggtaagtgccgtgtgtggttcccgcgggcctggcctctttacgggttatggcccttgcgtgccttgaattactgacactgacatccactttttctttttctccacag

SEQ ID NO: 18 - DNA, 6491 bp, synthetic construct
  source 1..6491: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcgccaccatgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgaggtaccgatcacatatgcctttaattaaacaactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctgtcgacggaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctatgtacatattatgatatagatacaacgtatgcaatggccaatagccaatattgatttatgctatataaccaatgaataatatggctaatggccaatattgacatgcattgggaggagaccgggacgagacgaaccctgataaatgcttcaataatattatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaataggcgcccttaaggctagctaga

SEQ ID NO: 19 - DNA, 35 bp, unidentified
  source 1..35: mol_type = genomic DNA
caggagtcaattttacgcagactatctttctaggg

SEQ ID NO: 20 - DNA, 471 bp, synthetic construct
  source 1..471: mol_type = other DNA
actaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggag

SEQ ID NO: 21 - DNA, 6623 bp, synthetic construct
  source 1..6623: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgtacccatacgatgttccagattacgcttctaccgccgtgctggaaaatcctggcctgggcagaaagctgagcgacttcggccaagagacaagctacatcgaggacaactgcaaccagaacggcgccatcagcctgatcttcagcctgaaagaagaagtgggcgccctggccaaggtgctgagactgttcgaagagaacgacgtgaacctgacacacatcgagagcagacccagcagactgaagaaggacgagtacgagttcttcacccacctggacaagcggagcctgcctgctctgaccaacatcatcaagatcctgcggcacgacatcggcgccacagtgcacgaactgagccgggacaagaaaaaggacaccgtgccatggttccccagaaccatccaagagctggacagattcgccaaccagatcctgagctatggcgccgagctggacgctgatcaccctggcttcaaggaccccgtgtaccgggccagaagaaagcagtttgccgatatcgcctacaactaccggcacggccagcctattcctcgggtcgagtacatggaagaggaaaagaaaacctggggcaccgtgttcaagaccctgaagtccctgtacaagacccacgcctgctacgagtacaaccacatcttcccactgctcgagaagtactgcggcttccacgaggacaatatccctcagctcgaggacgtgtcccagttcctgcaaacctgcaccggctttagactgaggcctgttgccggactgctgagcagcagagattttctcggcggcctggccttcagagtgttccactgtacccagtacatcagacacggcagcaagcccatgtacacccctgagcctgatatctgccacgagctgctgggacatgtgcccctgttcagcgatagatccttcgcccagttcagccaagagatcggactggcttctctgggagcccctgacgagtacattgagaagctggccaccatctactggttcaccgtggagttcggcctgtgcaagcagggcgatagcatcaaggcttatggcgctggcctgctgtctagctttggcgagctgcagtactgtctgagcgagaagcctaagctgctgcccctggaactggaaaagaccgccatccagaactacaccgtgaccgagttccagcctctgtactacgtggccgagagcttcaacgacgccaaagaaaaagtgcggaacttcgccgccaccattcctcggcctttcagcgtccgctacgacccctacacacagcggatcgaggtgctggacaacacacagcagctgaaaattctggccgacagcatcaacagcgagatcggcatcctgtgcagcgccctgcagaaaatcaagtgaggtaccgatcacatatgcctttaattaaacactggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgtcatttagaaagagagagcaatatttcaagaatgcaggagtcaattttacgcagactatctttctagggttaatctagctagccttaagggcgctttcctgtcgacgactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctatgtacatattatgatatagatacaacgtatgcaatggccaatagccaatattgatttatgctatataaccaatgaataatatggctaatggccaatattgacatgcattgggaggagaccgggacgagacgaaccctgataaatgcttcaataatattatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaataggcgcccttaaggctagctagacctgcaggcag

SEQ ID NO: 22 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 23 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 24 - protein, 593 aa, synthetic construct
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 25 - protein, 593 aa, synthetic construct
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 26 - protein, 7 aa, synthetic construct
PKKKRKV

SEQ ID NO: 27 - DNA, 21 bp, synthetic construct
  source 1..21: mol_type = other DNA
cccaagaagaagcggaaagtt

SEQ ID NO: 28 - protein, 5 aa, synthetic construct
GGGGS

SEQ ID NO: 29 - DNA, 15 bp, synthetic construct
  source 1..15: mol_type = other DNA
ggcggcggaggcagc

SEQ ID NO: 30 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 31 - DNA, 1815 bp, synthetic construct
  source 1..1815: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 32 - protein, 603 aa, synthetic construct
PKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 33 - DNA, 1809 bp, synthetic construct
  source 1..1809: mol_type = other DNA
cccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 34 - protein, 340 aa, synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 35 - protein, 339 aa, synthetic construct
GKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 36 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 37 - protein, 339 aa, synthetic construct
GKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 38 - protein, 1496 aa, synthetic construct
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 39 - protein, 1495 aa, synthetic construct
SKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 40 - protein, 649 aa, synthetic construct
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 41 - protein, 648 aa, synthetic construct
EEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 42 - protein, 636 aa, synthetic construct
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 43 - protein, 635 aa, synthetic construct
MLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

(19) United States
(12) Patent Application Publication
(10) Pub. No.: US 2026/0083860 A1
(43) Pub. Date: Mar. 26, 2026

(54) COMPOSITIONS AND METHODS FOR GENOME EDITING

(71) Applicant: Poseida Therapeutics, Inc., San Diego, CA (US)

(72) Inventors: Oscar ALVAREZ, San Diego, CA (US); Ming-Yue LEE, San Diego, CA (US); Blair B. MADISON, San Diego, CA (US)

(21) Appl. No.: 19/303,777

(22) Filed: Aug. 19, 2025

Related U.S. Application Data
(63) Continuation of application No. PCT/US2024/016634, filed on Feb. 21, 2024.
(60) Provisional application No. 63/486,165, filed on Feb. 21, 2023.

(51) Int. Cl. A61K 48/00; A61K 9/1271; A61K 9/51; A61P 3/06; C12N 9/22; C12N 15/11; C12N 15/88

(52) U.S. Cl. CPC A61K 48/005; A61K 9/1271; A61K 9/5123; A61P 3/06; C12N 9/226; C12N 15/111; C12N 15/88; C07K 2319/09; C07K 2319/80; C12N 2310/20

(57) ABSTRACT

Disclosed are methods and compositions for functional genetic modifications at selected sites. Also provided are cell populations, which comprise targeted integration of one or more exogenous polynucleotides, and/or indels at one or more selected gene loci.

### RELATED APPLICATIONS

This application is a continuation of International Patent Application No. PCT/US2024/016634, filed Feb. 21, 2024, which claims benefit of U.S. Provisional Application No. 63/486,165, filed on Feb. 21, 2023. The contents of each application are incorporated herein by reference in their entireties.

### SEQUENCE LISTING

The instant application contains a Sequence Listing, which has been submitted electronically in XML file format, and is hereby incorporated by reference into the specification in its entirety. The XML file containing the Sequence Listing XML is named “000218-0135-101-SL.xml,” was created on Aug. 19, 2025, and is 70,541 bytes in size.

### FIELD

The present disclosure is directed to the field of genetic editing and genomic engineering. More particularly, the present disclosure is directed to compositions and methods for targeted genetic modification.

### BACKGROUND OF THE INVENTION

Genome editing refers to strategies and techniques for the targeted, specific modification of the genetic information (genome) of living organisms. Genome engineering is an active field of research because of the wide range of possible applications, particularly in the area of human health, e.g., to correct a gene carrying a harmful mutation or to explore the function of a gene. Early technologies developed to insert a transgene into a living cell were often limited by the random nature of the insertion location of the new sequence into the genome. Common genome editing strategies allow a specific area of the DNA to be modified, thereby increasing precision of the correction or insertion compared to earlier technologies. While these platforms offer a greater degree of reproducibility and decreased level of unintended effects from random insertions and deletions in the genome, limitations remain. There is a need to develop gene editing platforms and fusions proteins with superior efficacy in genome editing. There is also a need to develop safe and effective donor cells in support of cell therapy treatments involving regenerative medicine and or immuno-oncology related diseases using such gene editing platforms.

### SUMMARY OF THE INVENTION

The disclosure provides a polypeptide comprising the amino acid sequence of SEQ ID NO: 35. The disclosure also provides a polynucleotide encoding the polypeptide, a vector comprising the polypeptide and a pharmaceutical composition comprising the vector and at least one pharmaceutically acceptable excipient or diluent.

The disclosure also provides a method of modifying a target sequence in the genome of a plurality of cells comprising introducing to a population of unmodified cells a composition comprising: a) a polypeptide comprising the amino acid sequence of SEQ ID NO: 35, or a polynucleotide encoding the polypeptide of SEQ ID NO: 35; and b) at least one guide RNA (gRNA), thereby generating a modification at the target sequence in the genome, and wherein 1.1-fold to 50-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10.

In some aspects, 1.2-fold to 1.8-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10. In some aspects, 3-fold to 40-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10.

In some aspects, the modification at the target sequence in the genome is a deletion, a insertion, a substitution, a inversion and/or a relocation. In some aspects, the composition is encapsulated in at least one lipid nanoparticle comprising: about 40.75% of a compound of Formula (I) by moles,

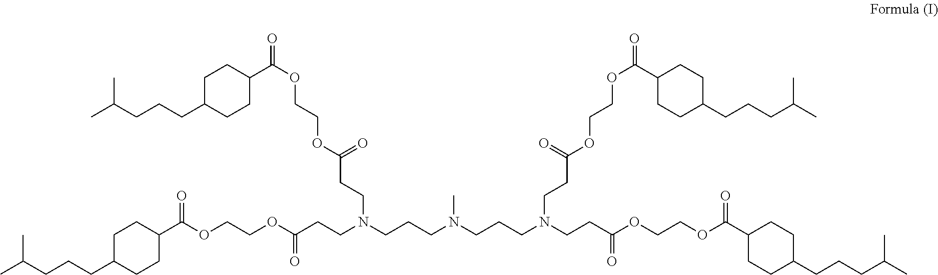 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

about 51.75% of cholesterol by moles, about 5% of DOPC by moles, and
about 2.5% of DMG-PEG2000 by moles; wherein a polynucleotide encoding the polypeptide of SEQ ID NO: 35 is an RNA molecule, and wherein the ratio of lipid to RNA molecule in the at least one nanoparticle is about 120:1 (w/w).

In some aspects, the plurality of cells comprise: (a) a liver cell, preferably wherein the liver cell is a hepatocyte, a hepatic stellate cell, Kupffer cell or liver sinusoidal endothelial cell; (b) a T-cell, preferably wherein the T-cell is an activated T-cell, a resting T-cell or a stem memory T cell (T_{SCM} cell); or (c) a hematopoietic stem cell (HSC).

The disclosure also provides a cell modified according to any one of the methods of the disclosure. The disclosure also provides a composition comprising a population of cells modified according to any one of the methods of the disclosure.

The disclosure also provides a method of treating at least one disease or disorder in a subject in need thereof comprising administering to the subject at least one therapeutically effective amount of the compositions, the pharmaceutical compositions or the cells described herein. In some aspects, the at least one disease or disorder is a liver disease or disorder, preferably wherein the liver disease or disorder is: (a) a metabolic liver disorder; (b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof. In some aspects, the at least one disease or disorder is cancer. In some aspects, the at least one disease or disorder is hemophilia A.

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### DETAILED DESCRIPTION OF THE INVENTION

The present invention provides a method for genetically modifying a genome to include a polynucleotide insertion, deletion and/or a substitution. In particular, the present disclosure overcomes problems associated with current technologies by providing a method for efficiently genetically modifying cellular genomes to include polynucleotide insertions, deletions and/or substitutions. This is advantageous for the modification of genes corresponding to disease phenotypes, which have important implications for therapeutic purposes. This is advantageous for providing higher yields of modified cells in comparison to current technologies, which is in turn advantageous for the production of cells for therapeutic use.

The present disclosure is based, at least in part, on the discovery that genetically modifying cells using a composition comprising mutants of Cas-CLOVER results in higher deletion rates, in comparison to wildtype Cas-CLOVER (NLS-dCas9-Clo051-NLS) or conventional CRISPR/Cas9 systems. Structural modeling and sequence analysis of the Clo051 endonuclease domain of Cas-CLOVER against other nuclease domains (e.g., FokI nuclease domain) revealed several conserved amino acid positions that yield hyperactivity, thereby providing functional enhancement. Serine to proline modifications at positions within the conserved alpha-helix-loop result in stabilization effect, as the proline changes the turn of the loop and reduces the degree of freedom at this end of the loop-helix. Additionally, the proline can still interact with phosphate backbone moieties of the bound DNA within acceptable hydrogen-bond distance (e.g., approximately 5 Å or less), preserving the potential interactions that would have been observed for wildtype Clo051 endonuclease at this position. Use of a mutant Cas-CLOVER comprising a mutant Clo051 endonuclease or portions thereof results in functional enhancement, such as improvements to genome editing and knock-in and knock-out efficiency.

Accordingly, the present invention provides an efficient, reliable, and targeted approach for transiently or stably integrating one or more exogenous genes, and maintaining high viability and functional responses of the gene in various cell types. Non-limiting examples of cell types include expanded iPSC, as well as in differentiated cells derived from the modified iPSC, including but not limited to HSC (hematopoietic stem and progenitor cell), T cell progenitor cells, NK cell progenitor cells, T cells, NKT cells, NK cells. These are useful for the development of safe and effective universal donor cells in support of cell therapy treatments (e.g., regenerative medicine or treatment of immuno-oncology related diseases).

The present disclosure overcomes problems associated with current technologies by providing compositions comprising genetically engineered fusion molecules (e.g. mutants of Cas-CLOVER) for targeted reduction or elimination of gene products in a cell for use in in vivo gene therapy. The compositions comprising genetically engineered fusion molecules of the disclosure are useful for treatment of genetic diseases. Non-limiting examples include, diseases of the liver, diseases associated with hepatocytes, sickle cell disease, or beta thalassemia. Accordingly, methods of making genetically engineered fusion molecules and pharmaceutical formulations thereof (e.g., lipid nanoparticle formulations) for use in in vivo delivery are also provided.

The present disclosure is based, at least in part, on the discovery that even a modest fold increase of editing activity (e.g. range of 1.5 to 3-fold) provided by a mutant Cas-CLOVER of the disclosure relative to a wildtype Cas-CLOVER, can have a significant impact on in vivo therapeutic applications. This increase could translate into the difference between a non-useful therapeutic index of efficacy of 25% to 50%, and a useful therapeutic index of efficacy of 75%, where the majority of a diseased (mutant) or disease-relevant alleles can be inactivated, including bi-allelic editing. As a non-limiting example, for HBG locus targeting, the magnitude of the improvement provided by the mutant Cas-CLOVER of the disclosure, could cross a key therapeutic threshold to fully enable activation of fetal hemoglobin (HbF) expression, which would provide therapeutic efficacy for functional correction of sickle cell disease, or beta thalassemia. Alternatively, the increased activity of the mutant Cas-CLOVER can enable a decreased dosage of the administered therapeutic product, with benefits to tolerability and toxicity.

### Methods for Targeted Genome Editing at Selected Locus

### Gene Editing Compositions and Methods

A modified cell may be produced by introducing a transgene into the cell. The introducing step may comprise delivery of a nucleic acid sequence, a transgene, and/or a genomic editing construct via a non-transposition delivery system.

Introducing a nucleic acid sequence, transgene and/or a genomic editing construct into a cell ex vivo, in vivo, in vitro or in situ can comprise one or more of topical delivery, adsorption, absorption, electroporation, spin-fection, co-culture, transfection, mechanical delivery, sonic delivery, vibrational delivery, magnetofection or by nanoparticle-mediated delivery. Introducing a nucleic acid sequence, a transgene and/or a genomic editing construct into a cell ex vivo, in vivo, in vitro or in situ can comprise liposomal transfection, calcium phosphate transfection, fugene transfection, and dendrimer-mediated transfection. Introducing a nucleic acid sequence, a transgene, and/or a genomic editing construct into a cell ex vivo, in vivo, in vitro or in situ by mechanical transfection can comprise cell squeezing, cell bombardment, or gene gun techniques. Introducing a nucleic acid sequence, transgene and/or a genomic editing construct into a cell ex vivo, in vivo, in vitro or in situ by nanoparticle-mediated transfection can comprise liposomal delivery, delivery by micelles, and delivery by polymerosomes.

Introducing a nucleic acid sequence, transgene and/or a genomic editing construct into a cell ex vivo, in vivo, in vitro or in situ can comprise a non-viral vector. The non-viral vector can comprise a nucleic acid. The non-viral vector can comprise plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), double strandedoligonucleotides (dsODNs), single-stranded mRNA (ssRNA), and double-stranded mRNA (dsRNA). The non-viral vector can comprise a transposon as described herein.

Introducing a nucleic acid sequence, transgene and/or a genomic editing construct into a cell ex vivo, in vivo, in vitro or in situ can comprise a viral vector. The viral vector can be a non-integrating non-chromosomal vector. Non-limiting examples of non-integrating non-chromosomal vectors include adeno-associated virus (AAV), adenovirus, and herpes viruses. The viral vector can be an integrating chromosomal vector. Non-limiting examples of integrating chromosomal vectors include adeno-associated vectors (AAV), Lentiviruses, and gamma-retroviruses.

Introducing a nucleic acid sequence, transgene and/or a genomic editing construct into a cell ex vivo, in vivo, in vitro or in situ can comprise a combination of vectors. Non-limiting examples of vector combinations include viral and non-viral vectors, a plurality of non-viral vectors, or a plurality of viral vectors. Non-limiting examples of vector combinations include a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

The nucleic acid sequence or transgene can be about 1 kb to about 15 kb in size. The nucleic acid sequence or transgene can be at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb in size. The nucleic acid sequence or transgene can be about 1 kb, about 2 kb, about 3 kb, about 4 kb, about 5 kb, about 6 kb, about 7 kb, about 8 kb, about 9 kb, about 10 kb, about 11 kb, about 12 kb, about 13 kb, about 14 kb or about 15 kb in size.

Genome modification can comprise introducing a nucleic acid sequence, transgene and/or a genomic editing construct into a cell ex vivo, in vivo, in vitro or in situ to stably integrate a nucleic acid sequence. The stable chromosomal integration can be a random integration, a site-specific integration, or a biased integration. The site-specific integration can be non-assisted or assisted. The assisted site-specific integration is co-delivered with a site-directed nuclease. The site-directed nuclease comprises a transgene with 5′ and 3′ nucleotide sequence extensions that contain a percentage homology to upstream and downstream regions of the site of genomic integration. The transgene with homologous nucleotide extensions enable genomic integration by homologous recombination, microhomology-mediated end joining, or nonhomologous end-joining. The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

Enzymes can be used to create strand breaks in the host genome to facilitate delivery or integration of the transgene. Enzymes can create single-strand breaks or double-strand breaks. Non-limiting examples of break-inducing enzymes include transposases, integrases, endonucleases, CRISPR/Cas9, transcription activator-like effector nucleases (TALEN), zinc finger nucleases (ZFN), Cas-CLOVER™, and CPF1. Break-inducing enzymes can be delivered to the cell encoded in DNA, encoded in mRNA, as a protein, or as a nucleoprotein complex with a guide RNA (gRNA). Non-limiting examples of break-inducing enzymes are described in PCT/US2016/037922, PCT/US2018/066941, PCT/US2017/054799, each of which are incorporated by reference in their entirety. Exemplary mutant Cas-CLOVER break-inducing enzymes of the disclosure are also described below.

The site-specific transgene integration can be controlled by a vector-mediated integration site bias.

The site-specific transgene integration site can be a non-stable chromosomal insertion. The integrated transgene can be become silenced, removed, excised, or further modified. The genome modification can be a non-stable integration of a transgene. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In one aspect, the transient non-chromosomal insertion of a transgene does not integrate into a chromosome and the modified genetic material is not replicated during cell division.

The genome modification can be a semi-stable or persistent non-chromosomal integration of a transgene. A DNA vector encodes a Scaffold/matrix attachment region (S-MAR) module that binds to nuclear matrix proteins for episomal retention of a non-viral vector allowing for autonomous replication in the nucleus of dividing cells.

The genome modification can be a non-stable chromosomal integration of a transgene. The integrated transgene can become silenced, removed, excised, or further modified.

The modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. The modification to the genome by transgene insertion can occur via CRISPR/Cas9, TALEN, ZFNs, Cas-CLOVER™, and cpf1. Non-limiting examples of break-inducing enzymes are described in PCT/US2016/037922, PCT/US2018/066941, PCT/US2017/054799, each of which are incorporated by reference in their entirety. Exemplary mutant Cas-CLOVER break-inducing enzymes of the disclosure are also described herein.

In gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. An example recombinase that may be used as a cutting enzyme is the CRE recombinase. Non-limiting examples of integrases that may be used in insertion tools include viral based enzymes taken from any of a number of viruses including AAV, gamma retrovirus, and lentivirus. Examples transposons/retrotransposons that may be used in insertion tools are described in more detail herein.

The present disclosure provides a gene editing composition and/or a cell comprising the gene editing composition. The gene editing composition can comprise a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

Exemplary dCas9-Clo051 Fusion Proteins

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a small, Cas9 (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease. A Staphylococcus aureus Cas9 with an active catalytic site comprises the amino acid sequence of SEQ ID NO: 1.

The disclosure provides compositions comprising an inactivated, small, Cas9 (dSaCas9) operatively-linked to an effector. The disclosure provides a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease. A dSaCas9 comprises the amino acid sequence of SEQ ID NO: 2, which includes a D10A and a N580A mutation to inactivate the catalytic site.

The disclosure provides compositions comprising an inactivated Cas9 (dCas9) operatively-linked to an effector. The disclosure provides a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 3 or SEQ ID NO: 4.

An exemplary Clo051 nuclease domain comprises, consists essentially of or consists of, the amino acid sequence of SEQ ID NO: 5. In some aspects, the Clo051 nuclease domain comprises at least one amino acid substitution. In some aspects, the amino acid substitution is in the alpha-helix-loop domain of the Clo051 nuclease. In some aspects, the amino acid substitution is at position 37 of SEQ ID NO: 5.

An exemplary dCas9-Clo051 fusion protein can comprise, consist essentially of, or consist of, the amino acid sequence of SEQ ID NO: 6. The exemplary dCas9-Clo051 fusion protein can be encoded by a polynucleotide which comprises, consists essentially of, or consists of, the nucleic acid sequence of SEQ ID NO: 7. The nucleic acid encoding the dCas9-Clo051 fusion protein can be DNA or RNA.

An exemplary dCas9-Clo051 fusion protein can comprise, consist essentially of, or consist of, the amino acid sequence of SEQ ID NO: 8. The exemplary dCas9-Clo051 fusion protein can be encoded by a polynucleotide which comprises, consists essentially of, or consists of, the nucleic acid sequence of SEQ ID NO: 9. The nucleic acid encoding the dCas9-Clo051 fusion protein can be DNA or RNA.

An exemplary dCas9-Clo051 fusion protein of the disclosure may further comprise at least one nuclear localization sequence (NLS). In some embodiments, the dCas9-Clo051 fusion protein of the disclosure comprises at least two nuclear localization sequences. In some embodiments, the NLS is on the N′terminal end of the dCas9-Clo051 fusion protein (NLS-dCas9-Clo051). In some embodiments, the NLS is on the C-terminal end of the dCas9-Clo051 fusion protein (dCas9-Clo051-NLS). In some embodiments, the NLS is on the N′terminal end and at the C′terminal end of the dCas9-Clo051 fusion protein (“NLS-dCas9-Clo051-NLS” or “wildtype Cas-CLOVER”).

The NLS-dCas9-Clo051-NLS (“wildtype Cas-CLOVER”) fusion protein can comprise, consist essentially of, or consist of, the amino acid sequence of SEQ ID NO: 10.

### NLS-dCas9-Clo051-NLS Amino Acid Sequence (NLS Amino Acid Sequence is Bolded and Underlined)

(SEQ ID NO: 10)
MAPKKKRKVEGIKSNISLLKDELRGQISHISHEYLSLIDLAFDSKQNRLFEMKVLELLVNEYGFKGRHLGGSRKPDG
IVYSTTLEDNFGIIVDTKAYSEGYSLPISQADEMERYVRENSNRDEEVNPNKWWENFSEEVKKYYFVFISGSFKGKF
EEQLRRLSMTTGVNGSAVNVVNLLLGAEKIRSGEMTIEELERAMENNSEFILKYGGGGSDKKYSIGLAIGTNSVGWA
VITDEYKVPSKKFKVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKERGHFLIEGD
LNPDNSDVDKLFIQLVQTYNQLFEENPINASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVKLNREDLLRKQRT
FDNGSIPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVDLLEKTNRK
VTVKQLKEDYFKKIECFDSVEISGVEDRFNASLGTYHDLLKIIKDKDELDNEENEDILEDIVLTLTLFEDREMIEER
LKTYAHLFDDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGKSKKLKSVKELLGITIMERSSF
EKNPIDFLEAKGYKEVKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTLTNLGAPAAFKYED
TTIDRKRYTSTKEVLDATLIHQSITGLYETRIDLSQLGGDGSPKKKRKVSS.

The nucleic acid encoding the NLS-dCas9-Clo051-NLS (“wildtype Cas-CLOVER”) fusion protein can be DNA or RNA. In some embodiments, a NLS-dCas9-Clo051-NLS is encoded by an mRNA sequence comprising, consisting essentially of or consisting of SEQ ID NO: 11.

NLS-dCas9-Clo051-NLS mRNA Sequence (NLS Amino Acid Sequence is Bolded and Underlined)

(SEQ ID NO: 11)
atggcaccaaagaagaaaagaaaagtggagggcatcaagtcaaacatcagcctgctgaaagacgaactgcggggaca
gattagtcacatcagtcacgagtacctgtcactgattgatctggccttcgacagcaagcagaatagactgtttgaga
tgaaagtgctggaactgctggtcaacgagtatggcttcaagggcagacatctgggcgggtctaggaaacctgacggc
atcgtgtacagtaccacactggaagacaacttcggaatcattgtcgataccaaggcttattccgagggctactctct
gccaattagtcaggcagatgagatggaaaggtacgtgcgcgaaaactcaaatagggacgaggaagtcaaccccaata
agtggtgggagaatttcagcgaggaagtgaagaaatactacttcgtctttatctcaggcagcttcaaagggaagttt
gaggaacagctgcggagactgtccatgactaccggggtgaacggatctgctgtcaacgtggtcaatctgctgctggg
cgcagaaaagatcaggtccggggagatgacaattgaggaactggaacgcgccatgttcaacaattctgagtttatcc
tgaagtatggaggcgggggaagcgataagaaatactccatcggactggccattggcaccaattccgtgggctgggct
gtcatcacagacgagtacaaggtgccaagcaagaagttcaaggtcctggggaacaccgatcgccacagtatcaagaa
aaatctgattggagccctgctgttcgactcaggcgagactgctgaagcaacccgactgaagcggactgctaggcgcc
gatatacccggagaaaaaatcggatctgctacctgcaggaaattttcagcaacgagatggccaaggtggacgatagt
ttctttcaccgcctggaggaatcattcctggtggaggaagataagaaacacgagcggcatcccatctttggcaacat
tgtggacgaagtcgcttatcacgagaagtaccctactatctatcatctgaggaagaaactggtggactccaccgata
aggcagacctgcgcctgatctatctggccctggctcacatgatcaagttccgggggcattttctgatcgagggagat
ctgaaccctgacaattctgatgtggacaagctgttcatccagctggtccagacatacaatcagctgtttgaggaaaa
cccaattaatgcctcaggcgtggacgcaaaggccatcctgagcgccagactgtccaaatctaggcgcctggaaaacc
tgatcgctcagctgccaggagagaagaaaaacggcctgtttgggaatctgattgcactgtccctgggcctgacaccc
aacttcaagtctaattttgatctggccgaggacgctaagctgcagctgtccaaagacacttatgacgatgacctgga
taacctgctggctcagatcggcgatcagtacgcagacctgttcctggccgctaagaatctgagtgacgccatcctgc
tgtcagatattctgcgcgtgaacacagagattactaaggccccactgagtgcttcaatgatcaaaagatatgacgag
caccatcaggatctgaccctgctgaaggctctggtgaggcagcagctgcccgagaaatacaaggaaatcttctttga
tcagagcaagaatggatacgccggctatattgacggcggggcttcccaggaggagttctacaagttcatcaagccca
ttctggaaaagatggacggcaccgaggaactgctggtgaagctgaatcgggaggacctgctgagaaaacagaggaca
tttgataacggaagcatccctcaccagattcatctgggcgaactgcacgccatcctgcgacggcaggaggacttcta
cccatttctgaaggataaccgcgagaaaatcgaaaagatcctgaccttcagaatcccctactatgtggggcctctgg
cacggggaaatagtagatttgcctggatgacaagaaagtcagaggaaactatcaccccctggaacttcgaggaagtg
gtcgataaaggcgctagcgcacagtccttcattgaaaggatgacaaattttgacaagaacctgccaaatgagaaggt
gctgcccaaacacagcctgctgtacgaatatttcacagtgtataacgagctgactaaagtgaagtacgtcaccgaag
ggatgcgcaagcccgcattcctgtccggagagcagaagaaagccatcgtggacctgctgtttaagacaaatcggaaa
gtgactgtcaaacagctgaaggaagactatttcaagaaaattgagtgtttcgattcagtggaaatcagcggcgtcga
ggacaggtttaacgcctccctggggacctaccacgatctgctgaagatcatcaaggataaggacttcctggacaacg
aggaaaatgaggacatcctggaggacattgtgctgacactgactctgtttgaggatcgcgaaatgatcgaggaacga
ctgaagacttatgcccatctgttcgatgacaaagtgatgaagcagctgaaaagaaggcgctacaccggatggggacg
cctgagccgaaaactgatcaatgggattagagacaagcagagcggaaaaactatcctggactttctgaagtccgatg
gcttcgccaacaggaacttcatgcagctgattcacgatgactctctgaccttcaaggaggacatccagaaagcacag
gtgtctggccagggggacagtctgcacgagcatatcgcaaacctggccggcagccccgccatcaagaaagggattct
gcagaccgtgaaggtggtggacgaactggtcaaggtcatgggacgacacaaacctgagaacatcgtgattgagatgg
cccgcgaaaatcagacaactcagaagggccagaaaaacagtcgagaacggatgaagagaatcgaggaaggcatcaag
gagctggggtcacagatcctgaaggagcatcctgtggaaaacactcagctgcagaatgagaaactgtatctgtacta
tctgcagaatggacgggatatgtacgtggaccaggagctggatattaacagactgagtgattatgacgtggatgcca
tcgtccctcagagcttcctgaaggatgactccattgacaacaaggtgctgaccaggtccgacaagaaccgcggcaaa
tcagataatgtgccaagcgaggaagtggtcaagaaaatgaagaactactggaggcagctgctgaatgccaagctgat
cacacagcggaaatttgataacctgactaaggcagaaagaggaggcctgtctgagctggacaaggccggcttcatca
agcggcagctggtggagacaagacagatcactaagcacgtcgctcagattctggatagcagaatgaacacaaagtac
gatgaaaacgacaagctgatcagggaggtgaaagtcattactctgaaatccaagctggtgtctgactttagaaagga
tttccagttttataaagtcagggagatcaacaactaccaccatgctcatgacgcatacctgaacgcagtggtcggga
ccgccctgattaagaaataccccaagctggagtccgagttcgtgtacggagactataaagtgtacgatgtccggaag
atgatcgccaaatctgagcaggaaattggcaaggccaccgctaagtatttcttttacagtaacatcatgaatttctt
taagaccgaaatcacactggcaaatggggagatcagaaaaaggcctctgattgagaccaacggggagacaggagaaa
tcgtgtgggacaagggaagggattttgctaccgtgcgcaaagtcctgtccatgccccaagtgaatattgtcaagaaa
actgaagtgcagaccgggggattctctaaggagagtattctgcctaagcgaaactctgataaactgatcgcccggaa
gaaagactgggaccccaagaagtatggcgggttcgactctccaacagtggcttacagtgtcctggtggtcgcaaagg
tggaaaaggggaagtccaagaaactgaagtctgtcaaagagctgctgggaatcactattatggaacgcagctccttc
gagaagaatcctatcgattttctggaagccaagggctataaagaggtgaagaaagacctgatcattaagctgccaaa
atactcactgtttgagctggaaaacggacgaaagcgaatgctggcaagcgccggagaactgcagaagggcaatgagc
tggccctgccctccaaatacgtgaacttcctgtatctggctagccactacgagaaactgaaggggtcccctgaggat
aacgaacagaagcagctgtttgtggagcagcacaaacattatctggacgagatcattgaacagatttcagagttcag
caagagagtgatcctggctgacgcaaatctggataaagtcctgagcgcatacaacaagcaccgagacaaaccaatcc
gggagcaggccgaaaatatcattcatctgttcaccctgacaaacctgggcgcccctgcagccttcaagtattttgac
accacaatcgatcggaagagatacacttctaccaaagaggtgctggatgctaccctgatccaccagagtattaccgg
cctgtatgagacacgcatcgacctgtcacagctgggaggcgatgggagccccaagaaaaagcggaaggtgtctagtt
aatga.

### Exemplary Mutant Cas-CLOVER Fusion Proteins

In some aspects, NLS-dCas9-Clo051-NLS (“wildtype Cas-CLOVER”) comprises at least one amino acid substitution. In some aspects, the amino acid substitution is located in the Clo051 domain of the NLS-dCas9-Clo051-NLS.

In some aspects, the NLS-dCas9-Clo051-NLS of SEQ ID NO: 10 can comprise at least one substitution at amino acid position 44. In some aspects, the amino acid substitution is S44P.

An exemplary S44P mutant NLS-dCas9-Clo051-NLS (“S44P Cas-CLOVER” or “S44P CC” or “S44P”) fusion protein can comprise, consist essentially of, or consist of, the amino acid sequence of SEQ ID NO: 35. The S44P Cas-CLOVER fusion protein can be encoded by a polynucleotide which comprises, consists essentially of, or consists of, the nucleic acid sequence of SEQ ID NO: 36. The nucleic acid encoding the dCas9-Clo051 fusion protein can be DNA or RNA.

| S44P Cas-CLOVER amino acid sequence | |
|---|---|
| (SEQ ID NO: 35) | |
| MAPKKKRKVE GIKSNISLLK DELRGQISHI SHEYLSLIDL AFDPKQNRLF EMKVLELLVN | 60 |
| EYGFKGRHLG GSRKPDGIVY STTLEDNFGI IVDTKAYSEG YSLPISQADE MERYVRENSN | 120 |
| RDEEVNPNKW WENFSEEVKK YYFVFISGSF KGKFEEQLRR LSMTTGVNGS AVNVVNLLLG | 180 |
| AEKIRSGEMT IEELERAMFN NSEFILKYGG GGSDKKYSIG LAIGTNSVGW AVITDEYKVP | 240 |
| SKKFKVLGNT DRHSIKKNLI GALLFDSGET AEATRLKRTA RRRYTRRKNR ICYLQEIFSN | 300 |
| EMAKVDDSFF HRLEESFLVE EDKKHERHPI FGNIVDEVAY HEKYPTIYHL RKKLVDSTDK | 360 |
| ADLRLIYLAL AHMIKFRGHF LIEGDLNPDN SDVDKLFIQL VQTYNQLFEE NPINASGVDA | 420 |
| KAILSARLSK SRRLENLIAQ LPGEKKNGLF GNLIALSLGL TPNFKSNEDL AEDAKLQLSK | 480 |
| DTYDDDLDNL LAQIGDQYAD LFLAAKNLSD AILLSDILRV NTEITKAPLS ASMIKRYDEH | 540 |
| HQDLTLLKAL VRQQLPEKYK EIFFDQSKNG YAGYIDGGAS QEEFYKFIKP ILEKMDGTEE | 600 |
| LLVKLNREDL LRKQRTFDNG SIPHQIHLGE LHAILRRQED FYPFLKDNRE KIEKILTFRI | 660 |
| PYYVGPLARG NSRFAWMTRK SEETITPWNF EEVVDKGASA QSFIERMTNF DKNLPNEKVL | 720 |
| PKHSLLYEYF TVYNELTKVK YVTEGMRKPA FLSGEQKKAI VDLLFKTNRK VTVKQLKEDY | 780 |
| FKKIECFDSV EISGVEDRFN ASLGTYHDLL KIIKDKDFLD NEENEDILED IVLTLTLFED | 840 |
| REMIEERLKT YAHLFDDKVM KQLKRRRYTG WGRLSRKLIN GIRDKQSGKT ILDFLKSDGF | 900 |
| ANRNFMQLIH DDSLTFKEDI QKAQVSGQGD SLHEHIANLA GSPAIKKGIL QTVKVVDELV | 960 |
| KVMGRHKPEN IVIEMARENQ TTQKGQKNSR ERMKRIEEGI KELGSQILKE HPVENTQLQN | 1020 |
| EKLYLYYLQN GRDMYVDQEL DINRLSDYDV DAIVPQSFLK DDSIDNKVLT RSDKNRGKSD | 1080 |
| NVPSEEVVKK MKNYWRQLLN AKLITQRKFD NLTKAERGGL SELDKAGFIK RQLVETRQIT | 1140 |
| KHVAQILDSR MNTKYDENDK LIREVKVITL KSKLVSDFRK DFQFYKVREI NNYHHAHDAY | 1200 |
| LNAVVGTALI KKYPKLESEF VYGDYKVYDV RKMIAKSEQE IGKATAKYFF YSNIMNFFKT | 1260 |
| EITLANGEIR KRPLIETNGE TGEIVWDKGR DFATVRKVLS MPQVNIVKKT EVQTGGFSKE | 1320 |
| SILPKRNSDK LIARKKDWDP KKYGGFDSPT VAYSVLVVAK VEKGKSKKLK SVKELLGITI | 1380 |
| MERSSFEKNP IDFLEAKGYK EVKKDLIIKL PKYSLFELEN GRKRMLASAG ELQKGNELAL | 1440 |
| PSKYVNFLYL ASHYEKLKGS PEDNEQKQLF VEQHKHYLDE IIEQISEFSK RVILADANLD | 1500 |
| KVLSAYNKHR DKPIREQAEN IIHLFTLTNL GAPAAFKYFD TTIDRKRYTS TKEVLDATLI | 1560 |
| HQSITGLYET RIDLSQLGGD GSPKKKRKVSS | 1591 |
| S44P Cas-CLOVER nucleic acid sequence | |
| (SEQ ID NO: 36) | |
| atggctccca agaagaagcg gaaggtcgag ggcatcaaga gcaacatcag cctgctgaag | 60 |
| gacgagctga gaggccagat cagccacatc toccacgagt acctgagcct gatcgacctg | 120 |
| gccttcgacc ccaagcagaa ccggctgttc gagatgaagg tgctggaact gctggtcaac | 180 |
| gagtacggct tcaagggcag acacctcggc ggcagcagaa agcctgatgg catcgtgtac | 240 |
| agcaccacac tcgaggacaa cttcggcatc atcgtggaca ccaaggccta cagcgagggc | 300 |
| tacagcctgc ctatctctca ggccgacgag atggaaagat acgtgcgcga gaacagcaac | 360 |
| cgcgacgagg aagtgaaccc caacaagtgg tgggagaact tcagcgagga agtcaaaaag | 420 |
| tactacttcg tgttcatcag cggcagcttt aagggcaagt tcgaggaaca gctgcggcgg | 480 |
| ctgtctatga ccacaggcgt taacggcagc gccgtgaacg tggtcaatct gctgctgggc | 540 |
| gccgagaaga ttagaagcgg cgagatgacc atcgaggaac tggaacgggc catgttcaac | 600 |
| aacagcgagt tcatcctgaa gtacggcgga ggcggcagcg acaagaagta ctctatcgga | 660 |
| ctggccatcg gcaccaactc tgttggatgg gccgtgatca ccgacgagta caaggtgccc | 720 |
| agcaagaaat tcaaagtgct gggcaacacc gaccggcaca gcatcaagaa gaatctgatc | 780 |
| ggcgccctgc tgttcgactc tggcgaaaca gccgaagcca ccagactgaa gagaaccgcc | 840 |
| agacggcggt acaccagaag aaagaaccgg atctgctacc tgcaagagat cttcagcaac | 900 |
| gagatggcca aggtggacga cagcttcttc cacagactgg aagagtcctt cctggtggaa | 960 |
| gaggacaaga agcacgagcg gcaccccatc ttcggaaata tcgtggacga ggtggcctac | 1020 |
| cacgagaagt accccaccat ctaccacctg agaaagaaac tggtggacag caccgacaag | 1080 |
| gccgacctgc gactgatcta tctggccctg gctcacatga tcaagttccg gggccacttc | 1140 |
| ctgatcgagg gcgacctgaa tcctgacaac tccgacgtgg acaagctgtt catccagctg | 1200 |
| gtgcagacct acaatcagct gttcgaagag aatcccatca acgcctctgg cgtggacgcc | 1260 |
| aaagccatcc tgtctgccag actgagcaag agcagacggc tggaaaacct gatcgctcag | 1320 |
| ctgcccggcg agaagaagaa tggcctgttc ggcaacctga ttgccctgtc tctgggcctg | 1380 |
| acacctaact tcaagtccaa cttcgatctg gccgaggatg ccaaactgca gctgtccaag | 1440 |
| gacacctacg acgacgacct ggataacctg ctggcccaga tcggcgatca gtacgccgac | 1500 |
| ttgtttctgg ccgccaagaa cctgtctgac gccatcctgc tgagcgacat cctgagagtg | 1560 |
| aacaccgaga tcacaaaggc ccctctgagc gcctctatga tcaagagata cgacgagcac | 1620 |
| caccaggatc tgaccctgct gaaagctctc gtcaggcagc agctgccaga gaagtacaaa | 1680 |
| gagattttct tcgaccagag caagaacggc tacgccggct acattgatgg cggagccagc | 1740 |
| caagaggaat tctacaagtt catcaagccc atcctcgaga agatggacgg cacagaggaa | 1800 |
| ctgctcgtga agctgaacag agaggacctg ctgcggaagc agcggacctt cgacaatggc | 1860 |
| tctatccctc accagatcca cctgggagag ctgcacgcca ttctgcggag acaagaggac | 1920 |
| ttttacccat tcctgaagga caaccgggaa aagattgaga agatcctgac cttcaggatc | 1980 |
| ccctactacg tgggaccact ggccagaggc aatagcagat tcgcctggat gaccagaaag | 2040 |
| agcgaggaaa ccatcacacc ctggaacttc gaagaggtgg tggacaaggg cgccagcgct | 2100 |
| cagtccttca tcgagcggat gaccaatttc gacaagaatc tgcccaacga gaaagtgctg | 2160 |
| cccaagcact ccctgctgta cgagtacttc accgtgtaca acgagctgac caaagtgaaa | 2220 |
| tacgtgaccg agggaatgag aaagcccgcc tttctgtccg gcgagcagaa aaaggccatc | 2280 |
| gtggatctgc tgttcaagac caaccggaaa gtgaccgtga agcagctgaa agaggactac | 2340 |
| ttcaagaaaa tcgagtgctt cgactccgtg gaaatcagcg gcgtggaaga tcggttcaat | 2400 |
| gccagcctgg gcacatacca cgatctgctg aaaattatca aggacaagga cttcctggac | 2460 |
| aacgaggaaa acgaggacat ccttgaggac atcgtgctga ccctgacact gttcgaggac | 2520 |
| agagagatga tcgaggaaag gctgaaaaca tacgcccacc tgttcgacga caaagtcatg | 2580 |
| aagcaactga agcggcggcg ctacacaggc tggggcagac tgtctagaaa gctgatcaac | 2640 |
| ggcatccggg acaagcagtc cggcaagacc atcctggact ttctgaagtc cgacggcttc | 2700 |
| gccaacagaa acttcatgca gctgattcac gacgacagcc tcaccttcaa agaggacatt | 2760 |
| cagaaggccc aggtttccgg ccagggcgat tctctgcacg agcacattgc caatctggcc | 2820 |
| ggctctcccg ccattaagaa gggcattctg cagacagtga aagtggtgga tgagctggtc | 2880 |
| aaagtgatgg ggagacacaa gcccgagaac atcgtgatcg aaatggccag agagaaccag | 2940 |
| accacacaga agggccagaa gaactcccgc gagagaatga agcggatcga agagggaatc | 3000 |
| aaagagctgg ggagccagat cctgaaagaa caccccgtgg aaaacaccca gctgcagaac | 3060 |
| gagaagctgt acctgtacta cctccagaac ggccgggata tgtacgtgga ccaagagctg | 3120 |
| gacatcaacc gcctgagcga ctacgatgtg gacgctatcg tgccccagtc ttttctgaaa | 3180 |
| gatgactcca tcgacaacaa ggtgctgacc agaagcgata agaaccgggg caagagcgac | 3240 |
| aacgtgccct ctgaagaggt cgtgaagaag atgaagaact actggcgaca gctgctgaac | 3300 |
| gccaagctga ttacccagcg gaagttcgat aacctgacca aggccgagag aggcggcctg | 3360 |
| tctgaactgg ataaggccgg cttcatcaag agacagctgg tggaaacccg gcagatcacc | 3420 |
| aaacacgtgg cacagattct ggactcccgg atgaacacca aatacgatga gaacgacaaa | 3480 |
| ctgatccggg aagtgaaagt catcaccctg aagtccaagc tggtgtccga tttccggaag | 3540 |
| gatttccagt tctacaaagt gcgggaaatc aacaactacc atcacgccca cgacgcctac | 3600 |
| ctgaatgccg ttgttggaac agccctgatc aagaagtatc ccaagctgga aagcgagttc | 3660 |
| gtgtacggcg actacaaggt gtacgacgtg cggaagatga tcgccaagag cgagcaagag | 3720 |
| attggaaagg ctaccgccaa atacttcttc tactccaaca tcatgaactt tttcaagaca | 3780 |
| gagatcaccc tcgccaacgg cgagatcaga aagcggcctc tgatcgagac aaacggcgaa | 3840 |
| accggcgaga ttgtgtggga taagggcaga gactttgcca cagtgcggaa ggtgctcagc | 3900 |
| atgccccaag tgaatatcgt gaaaaagacc gaggtgcaga caggcggctt cagcaaagag | 3960 |
| tccattctgc ctaagcggaa ctccgacaag ctgatcgccc ggaagaagga ctgggacccc | 4020 |
| aagaaatacg gcggcttcga tagccctacc gtggcctatt ctgtgctggt ggtggccaaa | 4080 |
| gtggaaaagg gaaagtccaa gaagctcaag agcgtcaaag aactcctggg catcaccatc | 4140 |
| atggaacggt ccagcttcga gaagaaccct atcgactttc tggaagccaa gggctacaaa | 4200 |
| gaagtcaaga aggacctgat catcaagctc cccaagtaca gcctgttcga gctggaaaat | 4260 |
| ggccggaagc ggatgctggc ttctgctggc gaactgcaga agggaaacga actggccctg | 4320 |
| cctagcaaat atgtgaactt cctgtacctg gccagccact atgagaagct gaagggcagc | 4380 |
| cccgaggaca atgagcagaa gcagcttttc gtcgagcagc acaagcacta cctggacgag | 4440 |
| atcatcgagc agatctccga gttctccaag agagtgatcc tggccgacgc caacctggac | 4500 |
| aaggttctgt ccgcctacaa caagcaccgg gataagccca tcagagagca ggccgagaat | 4560 |
| atcatccacc tgtttaccct gaccaacctg ggagcccctg ccgccttcaa gtacttcgac | 4620 |
| accaccatcg accggaagcg ctacaccagc accaaagaag tgctggacgc cacactgatc | 4680 |
| caccagagca tcaccggcct gtacgagaca cggatcgatc tgtctcagct tggaggcgac | 4740 |
| ggcagcccta agaagaagag aaaggtttcc agctaataa | 4779 |

Shown below is an amino acid sequence alignment of wildtype Cas-CLOVER amino acid sequence (SEQ ID NO: 10) and mutant S44P Cas-CLOVER amino acid sequence (SEQ ID NO: 35).

| | |
|---|---|
| MAPKKKRKVEGIKSNISLLKDELRGQISHISHEYLSLIDLAFDSKQNRLFEMKVLELLVN | 60 |
| MAPKKKRKVEGIKSNISLLKDELRGQISHISHEYLSLIDLAFDPKQNRLFEMKVLELLVN | 60 |
| ******************************************* **************** | |
| EYGFKGRHLGGSRKPDGIVYSTTLEDNFGIIVDTKAYSEGYSLPISQADEMERYVRENSN | 120 |
| EYGFKGRHLGGSRKPDGIVYSTTLEDNFGIIVDTKAYSEGYSLPISQADEMERYVRENSN | 120 |
| ************************************************************ | |
| RDEEVNPNKWWENFSEEVKKYYFVFISGSFKGKFEEQLRRLSMTTGVNGSAVNVVNLLLG | 180 |
| RDEEVNPNKWWENFSEEVKKYYFVFISGSFKGKFEEQLRRLSMTTGVNGSAVNVVNLLLG | 180 |
| ************************************************************ | |
| AEKIRSGEMTIEELERAMENNSEFILKYGGGGSDKKYSIGLAIGTNSVGWAVITDEYKVP | 240 |
| AEKIRSGEMTIEELERAMENNSEFILKYGGGGSDKKYSIGLAIGTNSVGWAVITDEYKVP | 240 |
| ************************************************************ | |
| SKKFKVLGNTDRHSIKKNLIGALLFDSGETAEATRLKRTARRRYTRRKNRICYLQEIFSN | 300 |
| SKKFKVLGNTDRHSIKKNLIGALLFDSGETAEATRLKRTARRRYTRRKNRICYLQEIFSN | 300 |
| ************************************************************ | |
| EMAKVDDSFFHRLEESFLVEEDKKHERHPIFGNIVDEVAYHEKYPTIYHLRKKLVDSTDK | 360 |
| EMAKVDDSFFHRLEESFLVEEDKKHERHPIFGNIVDEVAYHEKYPTIYHLRKKLVDSTDK | 360 |
| ************************************************************ | |
| ADLRLIYLALAHMIKFRGHFLIEGDLNPDNSDVDKLFIQLVQTYNQLFEENPINASGVDA | 420 |
| ADLRLIYLALAHMIKFRGHFLIEGDLNPDNSDVDKLFIQLVQTYNQLFEENPINASGVDA | 420 |
| ************************************************************ | |
| KAILSARLSKSRRLENLIAQLPGEKKNGLFGNLIALSLGLTPNFKSNFDLAEDAKLQLSK | 480 |
| KAILSARLSKSRRLENLIAQLPGEKKNGLFGNLIALSLGLTPNFKSNFDLAEDAKLQLSK | 480 |
| ************************************************************ | |
| DTYDDDLDNLLAQIGDQYADLFLAAKNLSDAILLSDILRVNTEITKAPLSASMIKRYDEH | 540 |
| DTYDDDLDNLLAQIGDQYADLFLAAKNLSDAILLSDILRVNTEITKAPLSASMIKRYDEH | 540 |
| ************************************************************ | |
| HQDLTLLKALVRQQLPEKYKEIFFDQSKNGYAGYIDGGASQEEFYKFIKPILEKMDGTEE | 600 |
| HQDLTLLKALVRQQLPEKYKEIFFDQSKNGYAGYIDGGASQEEFYKFIKPILEKMDGTEE | 600 |
| ************************************************************ | |
| LLVKLNREDLLRKQRTFDNGSIPHQIHLGELHAILRRQEDFYPFLKDNREKIEKILTFRI | 660 |
| LLVKLNREDLLRKQRTFDNGSIPHQIHLGELHAILRRQEDFYPFLKDNREKIEKILTFRI | 660 |
| ************************************************************ | |
| PYYVGPLARGNSRFAWMTRKSEETITPWNFEEVVDKGASAQSFIERMTNFDKNLPNEKVL | 720 |
| PYYVGPLARGNSRFAWMTRKSEETITPWNFEEVVDKGASAQSFIERMTNFDKNLPNEKVL | 720 |
| ************************************************************ | |
| PKHSLLYEYFTVYNELTKVKYVTEGMRKPAFLSGEQKKAIVDLLFKTNRKVTVKQLKEDY | 780 |
| PKHSLLYEYFTVYNELTKVKYVTEGMRKPAFLSGEQKKAIVDLLFKTNRKVTVKQLKEDY | 780 |
| ************************************************************ | |
| FKKIECFDSVEISGVEDRFNASLGTYHDLLKIIKDKDFLDNEENEDILEDIVLTLTLFED | 840 |
| FKKIECFDSVEISGVEDRFNASLGTYHDLLKIIKDKDFLDNEENEDILEDIVLTLTLFED | 840 |
| ************************************************************ | |
| REMIEERLKTYAHLFDDKVMKQLKRRRYTGWGRLSRKLINGIRDKQSGKTILDFLKSDGF | 900 |
| REMIEERLKTYAHLFDDKVMKQLKRRRYTGWGRLSRKLINGIRDKQSGKTILDFLKSDGF | 900 |
| ************************************************************ | |
| ANRNFMQLIHDDSLTFKEDIQKAQVSGQGDSLHEHIANLAGSPAIKKGILQTVKVVDELV | 960 |
| ANRNFMQLIHDDSLTFKEDIQKAQVSGQGDSLHEHIANLAGSPAIKKGILQTVKVVDELV | 960 |
| ************************************************************ | |
| KVMGRHKPENIVIEMARENQTTQKGQKNSRERMKRIEEGIKELGSQILKEHPVENTQLQN | 1020 |
| KVMGRHKPENIVIEMARENQTTQKGQKNSRERMKRIEEGIKELGSQILKEHPVENTQLQN | 1020 |
| ************************************************************ | |
| EKLYLYYLQNGRDMYVDQELDINRLSDYDVDAIVPQSFLKDDSIDNKVLTRSDKNRGKSD | 1080 |
| EKLYLYYLQNGRDMYVDQELDINRLSDYDVDAIVPQSFLKDDSIDNKVLTRSDKNRGKSD | 1080 |
| ************************************************************ | |
| NVPSEEVVKKMKNYWRQLLNAKLITQRKEDNLTKAERGGLSELDKAGFIKRQLVETRQIT | 1140 |
| NVPSEEVVKKMKNYWRQLLNAKLITQRKFDNLTKAERGGLSELDKAGFIKRQLVETRQIT | 1140 |
| ************************************************************ | |
| KHVAQILDSRMNTKYDENDKLIREVKVITLKSKLVSDFRKDFQFYKVREINNYHHAHDAY | 1200 |
| KHVAQILDSRMNTKYDENDKLIREVKVITLKSKLVSDFRKDFQFYKVREINNYHHAHDAY | 1200 |
| ************************************************************ | |
| LNAVVGTALIKKYPKLESEFVYGDYKVYDVRKMIAKSEQEIGKATAKYFFYSNIMNFFKT | 1260 |
| LNAVVGTALIKKYPKLESEFVYGDYKVYDVRKMIAKSEQEIGKATAKYFFYSNIMNFFKT | 1260 |
| ************************************************************ | |
| EITLANGEIRKRPLIETNGETGEIVWDKGRDFATVRKVLSMPQVNIVKKTEVQTGGFSKE | 1320 |
| EITLANGEIRKRPLIETNGETGEIVWDKGRDFATVRKVLSMPQVNIVKKTEVQTGGFSKE | 1320 |
| ************************************************************ | |
| SILPKRNSDKLIARKKDWDPKKYGGFDSPTVAYSVLVVAKVEKGKSKKLKSVKELLGITI | 1380 |
| SILPKRNSDKLIARKKDWDPKKYGGFDSPTVAYSVLVVAKVEKGKSKKLKSVKELLGITI | 1380 |
| ************************************************************ | |
| MERSSFEKNPIDFLEAKGYKEVKKDLIIKLPKYSLFELENGRKRMLASAGELQKGNELAL | 1440 |
| MERSSFEKNPIDFLEAKGYKEVKKDLIIKLPKYSLFELENGRKRMLASAGELQKGNELAL | 1440 |
| ************************************************************ | |
| PSKYVNFLYLASHYEKLKGSPEDNEQKQLFVEQHKHYLDEIIEQISEFSKRVILADANLD | 1500 |
| PSKYVNFLYLASHYEKLKGSPEDNEQKQLFVEQHKHYLDEIIEQISEFSKRVILADANLD | 1500 |
| ************************************************************ | |
| KVLSAYNKHRDKPIREQAENIIHLFTLTNLGAPAAFKYFDTTIDRKRYTSTKEVLDATLI | 1560 |
| KVLSAYNKHRDKPIREQAENIIHLFTLTNLGAPAAFKYFDTTIDRKRYTSTKEVLDATLI | 1560 |
| ************************************************************ | |
| HQSITGLYETRIDLSQLGGDGSPKKKRKVSS | 1591 |
| HQSITGLYETRIDLSQLGGDGSPKKKRKVSS | 1591 |
| ******************************* | |

(NLS-dCas9-Clo051-NLS) SEQ ID NO: 10
(S44P_Cas-CLOVER) SEQ ID NO: 35

A cell comprising the gene editing composition can express the gene editing composition stably or transiently.

The transgene can comprise a sequence encoding for a therapeutic agent. The therapeutic agent can be a protein or an RNA that provides a therapeutic benefit when administered to a cell or a subject. The therapeutic agent can be a therapeutic protein or a therapeutic RNA. The therapeutic agent can be human beta-globin (HBB), T87Q human beta-globin (HBB T87Q), BAF chromatin remodeling complex subunit (BCL11A) shRNA, insulin like growth factor 2 binding protein 1 (IGF2BP1), interleukin 2 receptor gamma (IL2RG), alpha galactosidase A (GLA), alpha-L-idurondase (IDUA), iduronate 2-sulfatase (IDS), cystinosin lysosomal cysteine transporter (CTNS). The transgene can comprise a sequence of Factor VIII or Factor IX. The transgene can comprise a sequence encoding a chimeric antigen receptor (CAR). The transgene can comprise a sequence encoding a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical. In one aspect, the transgene can comprise a sequence for a CAR and a sequence for a CSR. In one aspect, the transgene comprising a CAR or a CSR specifically binds to BCMA, PSMA, MUC1-C, CD133, c-KIT, CD19 or CD20. The transgene can comprise a sequence encoding for an inducible proapoptotic polypeptide comprising (a) a ligand binding region, (b) a linker, and (c) a caspase polypeptide, wherein the inducible proapoptotic polypeptide does not comprise a non-human sequence. The transgene can be integrated into the genome of the HSC. The integration can be stable or transient.

Factor VIII (FVIII) deficiency leads to development of Hemophilia A. Factor IX (FIX) deficiency leads to development of Hemophilia B. Prior to the compositions and methods of the disclosure, the standard treatment for hemophilia B involved an infusion of recombinant FIX every 2 to 3 days, at an expense of approximately $250,000 per year. In sharp contrast to this standard treatment option, iPSCs of the disclosure can be differentiated into any cell type including HSCs and maintained in humans for several decades.

The guide RNA can comprise a sequence complementary to a target sequence within a genomic DNA sequence. The target sequence within a genomic DNA sequence can be a target sequence within a safe harbor site of a genomic DNA sequence. Exemplary target sequences include but are not limited to HBB, TRAC, B2M, TCRb, GAPDH or SOX17.

The guide RNA can comprise a sequence complementary to at least one target sequence on a transposon, plasmid or vector. In some aspects, the complementary sequence to the guide RNA on the transposon, plasmid or vector is located within the transgene for targeted nucleic acid insertion. In some aspects, the complementary sequence to the guide RNA on the transposon, plasmid or vector is located within the transgene for targeted nucleic acid insertion. In some aspects, the complementary sequence on the transposon, plasmid or vector facilitates binding of a gRNA which is bound to an effector molecule, thereby tethering all components. In some aspects, the effector molecule is Cas-CLOVER. In some aspects, the Cas-CLOVER further comprises at least one NLS sequence. In some aspects, the NLS sequence of the Cas-CLOVER facilitates localization of the tethered components to the nucleus. This promotes localization of all components required for gene editing into the nucleus (Cas-CLOVER, gRNA and transposon, plasmid or vector), thereby increasing efficiency of gene editing.

gRNAs

As used herein, the term “guide sequence” in the context of a Cas-CLOVER system or a CRISPR-Cas9 system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” and “single guide RNA” are used interchangeably herein to refer to RNA-based molecules that are capable of forming a complex with a Cas-CLOVER or a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA may encompass RNA-based molecules having one or more chemically modifications (e.g., by chemical linking two ribonucleotides or by replacement of one or more ribonucleotides with one or more deoxyribonucleotides), as described herein.

The term “target region”, “target sequence” or “protospacer” as used interchangeably herein refers to the region of the target gene to which the Cas-CLOVER system or the CRISPR/Cas9-based system targets. The Cas-CLOVER or the CRISPR/Cas9-based system may include at least one gRNA, wherein the gRNAs target different DNA sequences. The target DNA sequences may be overlapping. The Cas-CLOVER system may include at least two gRNAs, wherein the gRNAs target different DNA sequences. The target sequence or protospacer is followed by a PAM sequence at the 3′ end of the protospacer. Different Type II systems have differing PAM requirements. For example, the S. pyogenes Type II system uses an “NGG” sequence, where “N” can be any nucleotide.

The guide RNA or the guide RNA of a Cas-CLOVER protein or a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the Cas-CLOVER or the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

As described above, the Cas-CLOVER system and the CRISPR/Cas9 system utilizes targeting gRNA and a shuttling gRNA that provides the targeting of the Cas-CLOVER system and the CRISPR/Cas9-based system. The gRNA may be a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

In some embodiment, the gRNA targets a region upstream of the target gene (e.g., HBB, B2M, TRAC or GAPDH gene locus), e.g., between 0-1000 bp upstream of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the target gene.

In some embodiments, the gRNA targets a region downstream of a target gene (e.g., HBB, B2M, TRAC or GAPDH gene locus), e.g., between 0-1000 bp downstream of a target gene. In some embodiments, the gRNA targets a region between 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp downstream of the target gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp downstream of the target gene.

gRNA can be divided into a target binding region and a Cas9 binding region. The target binding region hybridizes with a target region in a target gene. Methods for designing such target binding regions are known in the art, see, e.g., Doench et al., Nat Biotechnol. (2014) 32:1262-7; and Doench et al., Nat Biotechnol. (2016) 34:184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

Exemplary sgRNAs of the disclosure include but are not limited to sequences for targeting HBB, B2M, TRAC or GAPDH gene locus. Exemplary sgRNAs of the disclosure also include but are not limited to sequences for targeting hemoglobin, albumin, TTR, APOC3 and PCSK9. Exemplary sgRNAs of the disclosure comprise, consist essentially of or consists of the sequences as shown in Table 1.

TABLE 1
Exemplary sgRNAs of the disclosure

| Exemplary | | |
|---|---|---|
| sgRNA | Sequence | SEQ ID NO: |
| HBB L sgRNA | CUCAGGAGUCAGAUGCACCA | SEQ ID NO: 12 |
| HBB R sgRNA | GUGAACGUGGAUGAAGUUGG | SEQ ID NO: 13 |
| HBB L2 sgRNA | UGCACCAUGGUGUCUGUUUG | SEQ ID NO: 14 |
| HBB R2 sgRNA | CCUGUGGGGCAAGGUGAACG | SEQ ID NO: 15 |
| PCSK9-L gRNA | CCUGGGCACCAGCGCCGGUG | SEQ ID NO: 59 |
| PCSK9-R gRNA | GAUGCUCGCCCUCCCGUCCC | SEQ ID NO: 60 |
| Alb4-73406457 | GAGGCCUAGAUAGGCAUAUG | SEQ ID NO: 61 |
| gRNA | | |
| Alb4-73406506 | UGAAUAGGCUUUCUGGAUAA | SEQ ID NO: 62 |
| gRNA | | |
| APOC3-9_i3- | GGCAUUAGCUGGCAUAGCAG | SEQ ID NO: 63 |
| pair190-R | | |
| APOC3-9_i3- | GGGGAGAGGAGAGUACTGAU | SEQ ID NO: 64 |
| pair190-L | | |
| TTR-1 i4- | CCAGCUCCAUAGUUGAAUGU | SEQ ID NO: 65 |
| pair60-L | | |
| TTR-1 i4- | AGUUGCCUAGUGUUUGUUGC | SEQ ID NO: 66 |
| pair60-R | | |
| HBG1/2_HBG-L | CAAGGCUAUUGGUCAAGGCA | SEQ ID NO: 67 |
| HBG1/2_HBG-R | UAGUCUUAGAGUAUCCAGUG | SEQ ID NO: 68 |

Gene editing compositions, including Cas-CLOVER, and methods of using these compositions for gene editing are described in detail in PCT Application Numbers PCT/US2016/037922, PCT/US2018/066941, PCT/US2017/054799, U.S. Patent Publication Nos. 2017/0107541, 2017/0114149, 2018/0187185 and U.S. Pat. No. 10,415,024, each of which are incorporated herein by reference in its entirety. Exemplary gene editing compositions including mutant Cas-CLOVER and methods of using these compositions for gene editing are described herein.

Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)-containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a nuclease using the poly(histidine)-based micelles. Without wishing to be bound by a particular theory, it is believed that believed that in the micelles that are formed by the various triblock copolymers, the hydrophobic blocks aggregate to form a core, leaving the hydrophilic blocks and poly(histidine) blocks on the ends to form one or more surrounding layer.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example tri-block copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(¿-caprolactone) (PCL), and poly(trimethylene carbonate) (PTMC). Polymeric micelles comprised of 100% PEGylated surfaces possess improved in vitro chemical stability, augmented in vivo bioavailablity, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589, each of which are incorporated herein by reference in its entirety.

Gene editing compositions (e.g. mutant Cas-CLOVER) can also be delivered to cells using one or more lipid nanoparticle compositions comprising terpene lipidoid compounds and methods of making the same, as described in PCT Application No. PCT/US2023/061005, which is incorporated herein by reference in its entirety.

In some aspects, the composition is encapsulated in at least one lipid nanoparticle comprising: about 40.75% of a terpene lipidoid compound (e.g., HMA-404) by moles, about 51.75% of cholesterol by moles, about 5% of DOPC by moles, and about 2.5% of DMG-PEG2000 by moles, wherein a polynucleotide encoding the mutant Cas-CLOVER is an RNA molecule, and wherein the ratio of lipid to RNA molecule in the at least one nanoparticle is about 120:1 (w/w).

In some aspects, the terpene lipidoid compound is HMA-404 with the structure as shown below:

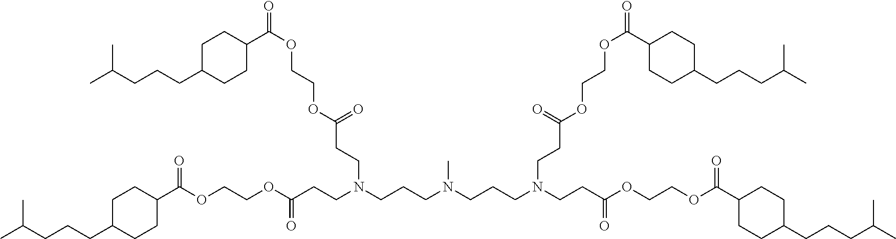 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Accordingly, in some aspects, the gene editing composition is encapsulated in at least one lipid nanoparticle comprising: about 40.75% of HMA-404 by moles, about 51.75% of cholesterol by moles, about 5% of DOPC by moles, and about 2.5% of DMG-PEG2000 by moles, wherein a polynucleotide encoding the mutant Cas-CLOVER polypeptide (e.g. the polypeptide of SEQ ID NO: 35) is an RNA molecule, and wherein the ratio of lipid to RNA molecule in the at least one nanoparticle is about 120:1 (w/w).

### Transposition Systems

The present disclosure also provides a composition comprising a transposon. In a preferred aspect, the composition comprising the transposon further comprises a plasmid comprising a nucleotide sequence encoding a transposase. The nucleotide sequence encoding the transposase may be a DNA sequence or an RNA sequence. Preferably, the sequence encoding the transposase is an mRNA sequence.

A transposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. The sequence encoding the SPB transposase is an mRNA sequence.

A transposon of the present disclosure can be a Footprint-Free™ transposon. In some aspects the transposase is a PBx transposase. The sequence encoding the PBx transposase is an mRNA sequence. In some aspects, the PBx transposase facilitates a Footprint-Free™ removal of a nucleic acid cassette in the transposon, plasmid or vector.

Non-limiting examples of PB transposons and PB, PBL and SPB transposases are described in detail in U.S. Pat. Nos. 6,218,182; 6,962,810; 8,399,643 and PCT Publication Nos. WO 2010/099296, WO 2010/099301, WO 2013/012824 each of which are incorporated herein in their entirety.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequence for one or more PB, PBL and SPB transposases are disclosed in U.S. Pat. Nos. 6,218,185; 6,962,810 and 8,399,643.

As described herein, in certain embodiments, the present invention features integration defective piggyBac transposons. Integration defective is meant to refer to a transposon that integrates at a lower frequency into the host genome than a corresponding wild type transposon. In certain exemplary embodiments, the inventive transposons integrate by conventional integration mechanisms.

Integration defective piggyBac transposons, in certain exemplary embodiments, are derived from the wildtype piggyBac sequence, SEQ ID NO: 16. In exemplary embodiments, the integration defective piggyBac transposon comprises a change in SEQ ID NO: 16 selected from R372A or K375A. In certain preferred embodiments, the integration defective piggyBac transposon comprises an amino acid sequence selected from SEQ ID NO: 17, SEQ ID NO: 18 or SEQ ID NO: 19. In certain embodiments, the amino acid change in SEQ ID NO: 16 comprises R372A and corresponds to SEQ ID NO: 17. The integration defective variant encoded by SEQ ID NO: 17 corresponds to a nucleotide change of CGA to GCA in SEQ ID NO: 20, and corresponds to SEQ ID NO: 21. In other certain embodiments, the amino acid change in SEQ ID NO: 16 comprises K375A and corresponds to SEQ ID NO: 18. The integration defective variant encoded by SEQ ID NO: 18 corresponds to a nucleotide change of AAA to GCA in SEQ ID NO: 20, and corresponds to SEQ ID NO: 50. In other certain embodiments, the amino acid change in SEQ ID NO: 2 comprises R372A, K375A and corresponds to SEQ ID NO: 19. The integration defective variant encoded by SEQ ID NO: 19 corresponds to a nucleotide change of CGA to GCA/AAA to GCA in SEQ ID NO: 20, and corresponds to SEQ ID NO: 22.

In exemplary embodiments, the integration defective piggyBac transposase comprises a change in SEQ ID NO: 16 at least selected from R372A or K375A and D450N. In some aspects, the PBx transposase comprises or consists of an amino acid sequence at least 75%, 80%, 85%, 90%, 95%, 96%, 97%, 98%, 99% or 100% (or any percentage in between) identical to SEQ ID NO: 23.

In exemplary embodiments, the integration defective piggyBac transposase comprises a change in SEQ ID NO: 16 at least selected from R372A or K375A and D450N. In some aspects, the PBx transposase comprises or consists of an amino acid sequence at least 75%, 80%, 85%, 90%, 95%, 96%, 97%, 98%, 99% or 100% (or any percentage in between) identical to SEQ ID NO: 24.

In some aspects, the PB transposase comprises or consists of an amino acid sequence at least 75%, 80%, 85%, 90%, 95%, 96%, 97%, 98%, 99% or 100% (or any percentage in between) identical to SEQ ID NO: 25.

The PB or PBL transposase can comprise or consist of an amino acid sequence having an amino acid substitution at two or more, at three or more or at each of positions 30, 165, 282, or 538 of the sequence of SEQ ID NO: 25. The transposase can be a SPB transposase that comprises or consists of the amino acid sequence of the sequence of SEQ ID NO: 25 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 75%, 80%, 85%, 90%, 95%, 96%, 97%, 98%, 99% or 100% (or any percentage in between) identical to SEQ ID NO: 26.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 25 or SEQ ID NO: 26 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

A transposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase. In a preferred aspect, the Sleeping Beauty transposase comprises or consists of an amino acid sequence at least 75%, 80%, 85%, 90%, 95%, 96%, 97%, 98%, 99% or 100% (or any percentage in between) identical to SEQ ID NO: 27. In a preferred aspect, hyperactive Sleeping Beauty (SB100X) transposase comprises or consists of an amino acid sequence at least 75%, 80%, 85%, 90%, 95%, 96%, 97%, 98%, 99% or 100% (or any percentage in between) identical to SEQ ID NO: 28.

A transposon of the present disclosure can be a Helraiser transposon. An exemplary Helraiser transposon includes Helibat1, which comprises or consists of a nucleic acid sequence at least 75%, 80%, 85%, 90%, 95%, 96%, 97%, 98%, 99% or 100% (or any percentage in between) identical to SEQ ID NO: 29. In some aspects, when the transposon is a Helraiser transposon, the transposase is a Helitron transposase (for example, as disclosed in WO 2019/173636). In a preferred aspect, Helitron transposase comprises or consists of an amino acid sequence at least 75%, 80%, 85%, 90%, 95%, 96%, 97%, 98%, 99% or 100% (or any percentage in between) identical to SEQ ID NO: 30.

A transposon of the present disclosure can be a Tol2 transposon. An exemplary Tol2 transposon, including inverted repeats, subterminal sequences and the Tol2 transposase, comprises or consists of a nucleic acid sequence at least 75%, 80%, 85%, 90%, 95%, 96%, 97%, 98%, 99% or 100% (or any percentage in between) identical to SEQ ID NO: 31. In some aspects, when the transposon is a Tol2 transposon, the transposase is a Tol2 transposase (for example, as disclosed in WO 2019/173636). In a preferred aspect, Tol2 transposase comprises or consists of an amino acid sequence at least 75%, 80%, 85%, 90%, 95%, 96%, 97%, 98%, 99% or 100% (or any percentage in between) identical to SEQ ID NO: 32.

A transposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. In a preferred aspect, a TcBuster transposase comprises or consists of an amino acid sequence at least 75%, 80%, 85%, 90%, 95%, 96%, 97%, 98%, 99% or 100% (or any percentage in between) identical to SEQ ID NO: 33. The polynucleotide encoding a TcBuster transposase can comprise or consist of a naturally occurring nucleic acid sequence or a non-naturally occurring nucleic acid sequence. In a preferred aspect, a TcBuster transposase is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 34.

In some aspects, a mutant TcBuster transposase comprises one or more sequence variations when compared to a wild type TcBuster transposase as described in more detail in PCT Publication No. WO 2019/173636 and PCT/US2019/049816.

The transposon can be a nanotransposon. A nanotransposon can comprise, consist essential of, or consist of (a) a sequence encoding a transposon insert, comprising a sequence encoding a first inverted terminal repeat (ITR), a sequence encoding a second inverted terminal repeat (ITR), and an intra-ITR sequence; (b) a sequence encoding a backbone, wherein the sequence encoding the backbone comprises a sequence encoding an origin of replication having between 1 and 450 nucleotides, inclusive of the endpoints, and a sequence encoding a selectable marker having between 1 and 200 nucleotides, inclusive of the endpoints, and (c) an inter-ITR sequence. In some aspects, the inter-ITR sequence of (c) comprises the sequence of (b). In some aspects, the intra-ITR sequence of (a) comprises the sequence of (b).

The selectable marker having between 1 and 200 nucleotides, inclusive of the endpoints, can comprise a sequence encoding a sucrose-selectable marker. The sequence encoding a sucrose-selectable marker can comprise a sequence encoding an RNA-OUT sequence. The sequence encoding an RNA-OUT sequence can comprise or consist of 137 base pairs (bp). The selectable marker having between 1 and 200 nucleotides, inclusive of the endpoints, can comprise a sequence encoding a fluorescent marker. The selectable marker having between 1 and 200 nucleotides, inclusive of the endpoints, can comprise a sequence encoding a cell surface marker.

The sequence encoding an origin of replication having between 1 and 450 nucleotides, inclusive of the endpoints, can comprise a sequence encoding a mini origin of replication. In some aspects, the sequence encoding an origin of replication having between 1 and 450 nucleotides, inclusive of the endpoints, comprises a sequence encoding an R6K origin of replication. The R6K origin of replication can comprise an R6K gamma origin of replication. The R6K origin of replication can comprise an R6K mini origin of replication. The R6K origin of replication can comprise an R6K gamma mini origin of replication. The R6K gamma mini origin of replication can comprise or consist of 281 base pairs (bp).

In some aspects of the nanotransposon, the sequence encoding the backbone does not comprise a recombination site, an excision site, a ligation site or a combination thereof. In some aspects, neither the nanotransposon nor the sequence encoding the backbone comprises a product of a recombination site, an excision site, a ligation site or a combination thereof. In some aspects, neither the nanotransposon nor the sequence encoding the backbone is derived from a recombination site, an excision site, a ligation site or a combination thereof.

In some aspects of the nanotransposon, a recombination site comprises a sequence resulting from a recombination event. In some aspects, a recombination site comprises a sequence that is a product of a recombination event. In some aspects, the recombination event comprises an activity of a recombinase (e.g., a recombinase site).

In some aspects of the nanotransposon, the sequence encoding the backbone does not further comprise a sequence encoding foreign DNA.

In some aspects of the nanotransposon, the inter-ITR sequence does not comprise a recombination site, an excision site, a ligation site or a combination thereof. In some aspects, the inter-ITR sequence does not comprise a product of a recombination event, an excision event, a ligation event or a combination thereof. In some aspects, the inter-ITR sequence is not derived from a recombination event, an excision event, a ligation event or a combination thereof. In some aspects, the inter-ITR sequence comprises a sequence encoding foreign DNA. In some aspects, the intra-ITR sequence comprises at least one sequence encoding an insulator and a sequence encoding a promoter capable of expressing an exogenous sequence in a mammalian cell. The mammalian cell can be a human cell. In some aspects, the intra-ITR sequence comprises a first sequence encoding an insulator, a sequence encoding a promoter capable of expressing an exogenous sequence in a mammalian cell and a second sequence encoding an insulator. In some aspects, the intra-ITR sequence comprises a first sequence encoding an insulator, a sequence encoding a promoter capable of expressing an exogenous sequence in a mammalian cell, a polyadenosine (polyA) sequence and a second sequence encoding an insulator. In some aspects, the intra-ITR sequence comprises a first sequence encoding an insulator, a sequence encoding a promoter capable of expressing an exogenous sequence in a mammalian cell, at least one exogenous sequence, a polyadenosine (polyA) sequence and a second sequence encoding an insulator.

Nanotransposons are described in more detail in PCT/US2019/067758, which is incorporated herein by reference in its entirety.

### Vector Systems

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses comprise two or more inverted terminal repeat (ITR) sequences located in cis next to a sequence encoding an scFv or a CAR of the disclosure. Exemplary adeno-associated viruses and recombinant adeno-associated viruses include, but are not limited to all serotypes (e.g., AAV1, AAV2, AAV3, AAV4, AAV5, AAV6, AAV7, AAV8, and AAV9). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03.

A vector of the present disclose can be a nanoparticle. Non-limiting examples of nanoparticle vectors include nucleic acids (e.g., RNA, DNA, synthetic nucleotides, modified nucleotides or any combination thereof), amino acids (L-amino acids, D-amino acids, synthetic amino acids, modified amino acids, or any combination thereof), polymers (e.g., polymersomes), micelles, lipids (e.g., liposomes), organic molecules (e.g., carbon atoms, sheets, fibers, tubes), inorganic molecules (e.g., calcium phosphate or gold) or any combination thereof. A nanoparticle vector can be passively or actively transported across a cell membrane.

The cell delivery compositions (e.g., transposons, vectors) disclosed herein can comprise a nucleic acid encoding a therapeutic protein or therapeutic agent. Examples of therapeutic proteins include those disclosed in PCT Publication No. WO 2019/173636 and PCT/US2019/049816.

### Nucleic Acid Molecules

Nucleic acid molecules of the disclosure can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one scFv; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for a specific scFv of the present disclosure. See, e.g., Ausubel, et al., supra, and such nucleic acid variants are included in the present disclosure.

As indicated herein, nucleic acid molecules of the disclosure can include, but are not limited to, those encoding the amino acid sequence of a scFv fragment, by itself; the coding sequence for the entire protein scaffold or a portion thereof; the coding sequence for a scFv, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

### Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides can be used for isolating, detecting, and/or quantifying nucleic acids comprising such polynucleotides. For example, polynucleotides of the present disclosure can be used to identify, isolate, or amplify partial or full-length clones in a deposited library. The polynucleotides can be genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of a protein scaffold encoded by the polynucleotides described herein. The polynucleotides embrace nucleic acid sequences that can be employed for selective hybridization to a polynucleotide encoding a protein scaffold of the present disclosure. See, e.g., Ausubel, supra, Colligan, supra, each entirely incorporated herein by reference.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygß gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

scFv Purification

An scFv can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

An scFv of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, from spontaneous or mutations and/or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-49-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, (CH2)3-, —NH—(CH2)6-NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimide derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnol. Bioeng., 56 (4): 456-463 (1997)), and the methods described in Hermanson, G. T., Bioconjugate Techniques, Academic Press: San Diego, Calif. (1996).

### Cells and Modified Cells of the Disclosure

Cells and modified cells of the disclosure can be mammalian cells. The cells and modified cells are human cells. Cells and modified cells of the disclosure can be immune cells. The immune cells of the disclosure can comprise iPSCs, lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts.

The immune precursor cells can comprise any cells which can differentiate into one or more types of immune cells. The immune precursor cells can comprise multipotent stem cells that can self-renew and develop into immune cells. The immune precursor cells can comprise hematopoietic stem cells (HSCs) or descendants thereof. The immune precursor cells can comprise precursor cells that can develop into immune cells. The immune precursor cells can comprise hematopoietic progenitor cells (HPCs).

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs can be isolated or derived from a primary or cultured stem cell. HSCs can be isolated or derived from an embryonic stem cell, a multipotent stem cell, a pluripotent stem cell, an adult stem cell, or an induced pluripotent stem cell (iPSC).

Immune precursor cells can comprise an HSC or an HSC descendent cell. Non-limiting examples of HSC descendent cells include multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes and macrophages.

HSCs produced by the disclosed methods can retain features of “primitive” stem cells that, while isolated or derived from an adult stem cell and while committed to a single lineage, share characteristics of embryonic stem cells. For example, the “primitive” HSCs produced by the disclosed methods retain their “stemness” following division and do not differentiate. Consequently, as an adoptive cell therapy, the “primitive” HSCs produced by the disclosed methods not only replenish their numbers, but expand in vivo. “Primitive” HSCs produced by disclosed the methods can be therapeutically-effective when administered as a single dose.

Primitive HSCs can be CD34+. Primitive HSCs can be CD34+ and CD38−. Primitive HSCs can be CD34+, CD38− and CD90+. Primitive HSCs can be CD34+, CD38−, CD90+ and CD45RA−. Primitive HSCs can be CD34+, CD38−, CD90+, CD45RA−, and CD49f+. Primitive HSCs can be CD34+, CD38−, CD90+, CD45RA−, and CD49f+.

Primitive HSCs, HSCs, and/or HSC descendent cells can be modified according to the disclosed methods to express an exogenous sequence (e.g., a chimeric antigen receptor or therapeutic protein). Modified primitive HSCs, modified HSCs, and/or modified HSC descendent cells can be forward differentiated to produce a modified immune cell including, but not limited to, a modified T cell, a modified natural killer cell and/or a modified B-cell.

The modified immune or immune precursor cells can be NK cells. The NK cells can be cytotoxic lymphocytes that differentiate from lymphoid progenitor cells. Modified NK cells can be derived from modified hematopoietic stem and progenitor cells (HSPCs) or modified HSCs. In some aspects, non-activated NK cells are derived from CD3-depleted leukapheresis (containing CD14/CD19/CD56+ cells).

The modified immune or immune precursor cells can be B cells. B cells are a type of lymphocyte that express B cell receptors on the cell surface. B cell receptors bind to specific antigens. Modified B cells can be derived from modified hematopoietic stem and progenitor cells (HSPCs) or modified HSCs.

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some embodiments, modified-T cells not only drive an initial response, but also persist in the patient as a stable population of viable memory T cells to prevent potential relapses. Alternatively, in some aspects, when it is not desired, the modified-T cells do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby Tx is the parent precursor cell that directly gives rise to T_{SCM}, which then, in turn, directly gives rise to T_{CM}, etc. Compositions of T cells of the disclosure can comprise one or more of each parental T cell subset with T_{SCM} cells being the most abundant (e.g., T_{SCM}>T_{CM}>T_{EM}>T_{E}>T_{TE}).

The immune cell precursor can be differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. The immune cell precursor can be a primitive HSC, an HSC, or a HSC descendent cell of the disclosure. The immune cell can be an early memory T cell, a stem cell like T-cell, a Naïve T cells (T_{N}), a T_{SCM}, a T_{CM}, a T_{EM}, a T_{E}, or a T_{TE}.

### Modified Cells of the Disclosure

The methods of the disclosure (e.g., using mutant Cas-CLOVER compositions) can modify and/or produce a population of modified immune cells, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, and wherein the modified cells have not been subjected to an enrichment protocol. In some embodiments, the modified cells are further enriched using an enrichment protocol.

The methods of the disclosure (e.g., using mutant Cas-CLOVER compositions) can modify and/or produce a population of modified cells having a modification at a target sequence, at a selected site in the genome. In some embodiments, the method of the disclosure can produce about 1.1 fold to about 50 fold greater population of modified cells having modification at a target sequence, at the selected site of the genome, in comparison to the number of modified cells that have not been generated using the method of the disclosure (e.g. using wildtype Cas-CLOVER or using CRISPR/Cas9 systems).

In some embodiments, the method of the disclosure can produce about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10 fold, about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold or about 45-fold to about 50-fold greater population of modified cells having the modification at a target sequence, at the selected site of the genome, in comparison to the number of modified cells that have not been generated using the method of the disclosure (e.g. using wildtype Cas-CLOVER or using CRISPR/Cas9 systems). In some embodiments, the method of the disclosure (e.g. using Cas-CLOVER systems) can produce about 1-fold, about 2-fold, about 3-fold, about 4-fold, about 5-fold, about 6-fold, about 7-fold, about 8-fold, about 9-fold, or about 10-fold greater population of modified cells having the modification at a target sequence, at the selected site of the genome, in comparison to the population of modified cells (e.g. immune cells or hepatocytes) that have not been generated using to the method of the disclosure (e.g. using CRISPR/Cas9 systems). In some embodiments, the method of the disclosure (e.g. using Cas-CLOVER systems) can produce about 1-fold, about 1.2-fold, about 1.3-fold, about 1.4-fold, about 1.5-fold, about 1.6-fold, about 1.7-fold, about 1.8-fold, about 1.9-fold, or about 2-fold greater population of modified cells having the transgene at the selected site of the genome, in comparison to the population of modified cells (e.g. immune cells or hepatocytes) that have not been generated using to the method of the disclosure (e.g. using CRISPR/Cas9 systems).

The methods of the disclosure (e.g., using mutant Cas-CLOVER compositions) can modify and/or produce a population of modified cells having a transgene or the sequence encoding the transgene at the selected site in the genome. In some embodiments, the method of the disclosure can produce about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10 fold greater population of modified cells having the transgene at the selected site of the genome, in comparison to the number of modified cells that have not been subjected to the method of the disclosure (e.g. using wildtype Cas-CLOVER or using CRISPR/Cas9 systems). In some embodiments, the method of the disclosure (e.g., using mutant Cas-CLOVER compositions) can produce about 1-fold, about 2-fold, about 3-fold, about 4-fold, about 5-fold, about 6-fold, about 7-fold, about 8-fold, about 9-fold, or about 10-fold greater population of modified cells having the transgene at the selected site of the genome, in comparison to the population of modified cells that have not been subjected to the method of the disclosure (e.g. using wildtype Cas-CLOVER or using CRISPR/Cas9 systems).

The methods of the disclosure (e.g., using mutant Cas-CLOVER compositions) can modify and/or produce a population of modified cells (e.g. immune cells or hepatocytes) having a transgene or the sequence encoding the transgene at the selected site in the genome. In some embodiments, the method of the disclosure (e.g. using mutant Cas-CLOVER compositions) can produce about 1-fold to about 2-fold, about 2-fold to about 3-fold, about 3-fold to about 4-fold, about 4-fold to about 5-fold, about 5-fold to about 6-fold, about 6-fold to about 7-fold, about 7-fold to about 8-fold, about 8-fold to about 9-fold, about 9-fold to about 10 fold greater population of viable modified cells, in comparison to the number of modified cells that have not been subjected to the method of the disclosure (e.g. using wildtype Cas-CLOVER or using CRISPR/Cas9 systems). In some embodiments, the method of the disclosure (e.g. using mutant Cas-CLOVER compositions) can produce about 1-fold, about 2-fold, about 3-fold, about 4-fold, about 5-fold, about 6-fold, about 7-fold, about 8-fold, about 9-fold, or about 10-fold greater population of viable modified cells, in comparison to the population of modified cells that have not been subjected to the method of the disclosure (e.g. using wildtype Cas-CLOVER or using CRISPR/Cas9 systems).

The methods of the disclosure can modify and/or produce a population of modified cells (e.g. immune cells or hepatocytes), wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, and wherein the modified cells have not been subjected to an enrichment protocol. In some embodiments, the modified cells are further enriched using an enrichment protocol.

A plurality of modified cells (e.g. immune cells or hepatocytes) of the population comprising a transgene or a sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, and wherein the modified cells have not been subjected to an enrichment protocol. In some embodiments, the modified cells are further enriched using an enrichment protocol.

Compositions and methods of producing and/or expanding the immune cells or immune precursor cells (e.g., the disclosed modified immune cells) and buffers for maintaining or enhancing a level of cell viability and/or a stem-like phenotype of the immune cells or immune precursor cells (e.g., the disclosed modified immune cells) are disclosed elsewhere herein.

Cells and modified immune cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

### Methods of Expressing a Chimeric Antigen Receptor

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface.

In some aspects, the cell population can comprise leukocytes and/or CD4+ and CD8+ leukocytes. The cell population can comprise CD4+ and CD8+ leukocytes in an optimized ratio. The optimized ratio of CD4+ to CD8+ leukocytes does not naturally occur in vivo. The cell population can comprise a tumor cell.

In some aspects, the conditions sufficient to transfer the CAR or the sequence encoding the CAR, transposon, or vector across a cell membrane of at least one cell in the cell population comprises at least one of an application of one or more pulses of electricity at a specified voltage, a buffer, and one or more supplemental factor(s). In some aspects, the conditions suitable for integration of the sequence encoding the CAR comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl₂, NazHPO₄, NAH₂PO₄, Sodium lactobionate, Mannitol, Sodium succinate, Sodium Chloride, CINa, Glucose, Ca(NO₃)₂, Tris/HCl, K₂HPO₄, KH₂PO₄, Polyethylenimine, Poly-ethylene-glycol, Poloxamer 188, Poloxamer 181, Poloxamer 407, Poly-vinylpyrrolidone, Pop313, Crown-5, or any combination thereof. The cell medium can comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the CAR, thereby generating an expanded cell population. The antigen can be presented on the surface of a substrate. The substrate can have any form, including, but not limited to a surface, a well, a bead or a plurality thereof, and a matrix. The substrate can further comprise a paramagnetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

A more detailed description of methods for expressing a CAR on the surface of a cell is disclosed in PCT Publication No. WO 2019/049816 and PCT/US2019/049816.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The composition can modify gene expression by decreasing gene expression. The composition can modify gene expression by transiently modifying gene expression (e.g., for the duration of binding of the ligand to the exogenous receptor). The composition can modify gene expression acutely (e.g., the ligand reversibly binds to the exogenous receptor). The composition can modify gene expression chronically (e.g., the ligand irreversibly binds to the exogenous receptor).

The exogenous receptor can comprise an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

The exogenous receptor can comprise a non-naturally occurring receptor. The non-naturally occurring receptor can be a synthetic, modified, recombinant, mutant or chimeric receptor. The non-naturally occurring receptor can comprise one or more sequences isolated or derived from a T-cell receptor (TCR). The non-naturally occurring receptor can comprise one or more sequences isolated or derived from a scaffold protein. In some aspects, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal. The non-naturally occurring receptor can comprise a transmembrane domain. The non-naturally occurring receptor can interact with an intracellular receptor that transduces an intracellular signal. The non-naturally occurring receptor can comprise an intracellular signaling domain. The non-naturally occurring receptor can be a chimeric ligand receptor (CLR). The CLR can be a chimeric antigen receptor (CAR).

The sequence encoding the inducible promoter of comprises a sequence encoding an NFκB promoter, a sequence encoding an interferon (IFN) promoter or a sequence encoding an interleukin-2 promoter. In some aspects, the IFN promoter is an IFNγ promoter. The inducible promoter can be isolated or derived from the promoter of a cytokine or a chemokine. The cytokine or chemokine can comprise IL2, IL3, IL4, IL5, IL6, IL10, IL12, IL13, IL17A/F, IL21, IL22, IL23, transforming growth factor beta (TGFβ), colony stimulating factor 2 (GM-CSF), interferon gamma (IFNγ), Tumor necrosis factor alpha (TNFα), LTα, perforin, Granzyme C (Gzmc), Granzyme B (Gzmb), C-C motif chemokine ligand 5 (CCL5), C-C motif chemokine ligand 4 (Ccl4), C-C motif chemokine ligand 3 (Ccl3), X-C motif chemokine ligand 1 (Xcl1) or LIF interleukin 6 family cytokine (Lif).

The inducible promoter can be isolated or derived from the promoter of a gene comprising a surface protein involved in cell differentiation, activation, exhaustion and function. In some aspects, the gene comprises CD69, CD71, CTLA4, PD-1, TIGIT, LAG3, TIM-3, GITR, MHCII, COX-2, FASL or 4-1BB.

The inducible promoter can be isolated or derived from the promoter of a gene involved in CD metabolism and differentiation. The inducible promoter can be isolated or derived from the promoter of Nr4a1, Nr4a3, Tnfrsf9 (4-1BB), Sema7a, Zfp3612, Gadd45b, Dusp5, Dusp6 and Neto2.

In some aspects, the inducible transgene construct comprises or drives expression of a signaling component downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of which are disclosed in PCT Publication No. WO 2019/173636 and PCT Application No. PCT/US2019/049816.

The present disclosure provides a method of producing a population of modified T-cells comprising, consisting essential of, or consisting of introducing into a plurality of primary human T-cells a composition comprising the CAR of the present disclosure or a sequence encoding the same to produce a plurality of modified T-cells. The present disclosure provides a composition comprising a population of modified T-cells produced by the method. In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population expresses the CAR of the present disclosure.

### Transposon and Vector Compositions

The present disclosure provides compositions and methods for delivering a therapeutic protein (antibody (e.g., scFv) or a CAR (e.g., comprising an scFv)) to a cell or a population of cells. Non-limiting examples of compositions for delivery of a composition of the disclosure to a cell or a population of cells include a transposon or a vector. Thus, the present disclosure provides a transposon comprising a therapeutic protein (an antibody (e.g., scFv) or a CAR (e.g., comprising an scFv)) or a vector comprising a therapeutic protein (an antibody (e.g., scFv) or a CAR (e.g., comprising an scFv)).

A transposon comprising a therapeutic protein of the disclosure or a vector comprising a therapeutic protein of the disclosure can further comprise a sequence encoding an inducible proapoptotic polypeptide. Alternatively, or in addition, one transposon or one vector can comprise a therapeutic protein of the disclosure and a second transposon or second vector can comprise a sequence encoding an inducible proapoptotic polypeptide of the disclosure. Inducible proapoptotic polypeptides are described in more detail herein.

A transposon comprising a therapeutic protein of the disclosure or a vector comprising a therapeutic protein of the disclosure can further comprise a sequence encoding a chimeric stimulatory receptor (CSR). Alternatively, or in addition, one transposon or one vector can comprise a CAR of the disclosure and a second transposon or a second vector can comprise a sequence encoding a CSR of the disclosure. Chimeric stimulatory receptors are described in more detail herein.

A transposon comprising a therapeutic protein of the disclosure or a vector comprising a therapeutic protein of the disclosure can further comprise a sequence encoding a recombinant HLA-E polypeptide. Alternatively, or in addition, one transposon or one vector can comprise a therapeutic protein of the disclosure and a second transposon or a second vector can comprise a sequence encoding a recombinant HLA-E polypeptide. Recombinant HLA-E polypeptide are described in more detail herein.

A transposon comprising a therapeutic protein of the disclosure or a vector comprising a therapeutic protein of the disclosure can further comprise a selection gene. The selection gene can encode a gene product essential for cell viability and survival. The selection gene can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCE, RAD51C (encoding RAD51 Paralog C), GCS (encoding glucosylceramide synthase), NKX2.2 (encoding NK2 Homeobox 2), or any combination thereof.

In a preferred aspect, the selection gene encodes a DHFR mutein enzyme. The DHFR mutein enzyme comprises, consists essentially of, or consists of the amino acid sequence of SEQ ID NO: 37. The DHFR mutein enzyme is encoded by a polynucleotide comprising, consisting essential of, or consisting of the nucleic acid sequence of SEQ ID NO: 38 or SEQ ID NO: 39. The amino acid sequence of the DHFR mutein enzyme can further comprise a mutation at one or more of positions 80, 113, or 153. The amino acid sequence of the DHFR mutein enzyme can comprise one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

A transposon comprising a CAR of the disclosure or a vector comprising a CAR of the disclosure can further comprise at least one self-cleaving peptide. For example, a self-cleaving peptide can be located between a CAR (e.g., comprising an scFv) and an inducible proapoptotic polypeptide; or, a self-cleaving peptide can be located between a CAR (e.g., comprising an scFv) and protein encoded by a selection gene.

A transposon comprising a CAR of the disclosure or a vector comprising a CAR of the disclosure can further comprise at least two self-cleaving peptides. For example, a first self-cleaving peptide is located upstream or immediately upstream of a CAR and a second self-cleaving peptide is located downstream or immediately downstream of a CAR; or, the first self-cleaving peptide and the second self-cleaving peptide flank a CAR. For example, a first self-cleaving peptide is located upstream or immediately upstream of an inducible proapoptotic polypeptide and a second self-cleaving peptide is located downstream or immediately downstream of an inducible proapoptotic polypeptide; or, the first self-cleaving peptide and the second self-cleaving peptide flank an inducible proapoptotic polypeptide. For example, a first self-cleaving peptide is located upstream or immediately upstream of protein encoded by a selection gene and a second self-cleaving peptide is located downstream or immediately downstream of a protein encoded by a selection gene; or, the first self-cleaving peptide and the second self-cleaving peptide flank a protein encoded by a selection gene.

### Inducible Proapoptotic Polypeptides

The inducible proapoptotic polypeptides disclosed herein are superior to existing inducible polypeptides because the inducible proapoptotic polypeptides of the disclosure are far less immunogenic. The inducible proapoptotic polypeptides are recombinant polypeptides, and, therefore, non-naturally occurring. Further, the sequences that are recombined to produce inducible proapoptotic polypeptides that do not comprise non-human sequences that the host human immune system could recognize as “non-self” and, consequently, induce an immune response in the subject receiving the inducible proapoptotic polypeptide, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain aspects, the non-human sequence comprises a restriction site. In certain aspects, the ligand binding region can be a multimeric ligand binding region. In certain aspects, the proapoptotic peptide is a caspase polypeptide. Non-limiting examples of caspase polypeptides include caspase 1, caspase 2, caspase 3, caspase 4, caspase 5, caspase 6, caspase 7, caspase 8, caspase 9, caspase 10, caspase 11, caspase 12, and caspase 14. Preferably, the caspase polypeptide is a caspase 9 polypeptide. The caspase 9 polypeptide can be a truncated caspase 9 polypeptide. Inducible proapoptotic polypeptides can be non-naturally occurring. When the caspase is caspase 9 or a truncated caspase 9, the inducible proapoptotic polypeptides can also be referred to as an “iC9 safety switch”.

An inducible caspase polypeptide can comprise (a) a ligand binding region, (b) a linker, and (c) a caspase polypeptide, wherein the inducible proapoptotic polypeptide does not comprise a non-human sequence. In certain aspects, an inducible caspase polypeptide comprises (a) a ligand binding region, (b) a linker, and (c) a truncated caspase 9 polypeptide, wherein the inducible proapoptotic polypeptide does not comprise a non-human sequence.

The ligand binding region can comprise a FK506 binding protein 12 (FKBP12) polypeptide. The amino acid sequence of the ligand binding region that comprises a FK506 binding protein 12 (FKBP12) polypeptide can comprise a modification at position 36 of the sequence. The modification can be a substitution of valine (V) for phenylalanine (F) at position 36 (F36V). The FKBP12 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 40. The FKBP12 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 41.

The linker region can comprise, consist essential of, or consist of, the amino acid sequence at least 75%, 80%, 85%, 90%, 95%, 96%, 97%, 98%, 99% or 100% (or any percentage in between) identical to SEQ ID NO: 42 or the linker region can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 43. In some aspects, the nucleic acid sequence encoding the linker does not comprise a restriction site.

The truncated caspase 9 polypeptide can comprise an amino acid sequence that does not comprise an arginine (R) at position 87 of the sequence. Alternatively, or in addition, the truncated caspase 9 polypeptide can comprise an amino acid sequence that does not comprise an alanine (A) at position 282 the sequence. The truncated caspase 9 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 44 or the truncated caspase 9 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 45.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 46 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 47.

In certain aspects when the polypeptide comprises a truncated caspase 9 polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 48 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 49.

Inducible proapoptotic polypeptides can be expressed in a cell under the transcriptional regulation of any promoter known in the art that is capable of initiating and/or regulating the expression of an inducible proapoptotic polypeptide in that cell.

Activation of inducible proapoptotic polypeptides can be accomplished through, for example, chemically induced dimerization (CID) mediated by an induction agent to produce a conditionally controlled protein or polypeptide. Proapoptotic polypeptides not only inducible, but the induction of these polypeptides is also reversible, due to the degradation of the labile dimerizing agent or administration of a monomeric competitive inhibitor.

Inducible proapoptotic peptides and methods of inducing these peptides are described in detail in U.S. Patent Publication No. WO 2019/0225667 and PCT Publication No. WO 2018/068022.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

### Methods of Using the Compositions of the Disclosure

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

The compositions of the disclosure can be used to treat a disease or disorder including, but not limited to: Osteopetrosis, Parkinson's Disease, Hunter Syndrome, Sickle Cell Disease, Severe Combined Immunodeficiency, Alpha-mannosidosis, Sideroblastic anemia, Autosomal Recessive Hyper IgE Syndrome, Primary Myelofibrosis, Cutaneous vasculitis, X-linked protoporphyria, Fucosidosis, Maroteaux Lamy syndrome, WAS Related Disorders, Chronic Granulomatous, Thalassemia Major, Hereditary Angioedema, Hereditary Lymphedemia, Hyper IgM Syndrome, Friedrich's Ataxia, Charcot Marie Tooth Disease, Phenylketonuria, Methylmalonic Acidemia, Adrenoleukodystrophy, Kugelberg Welander Syndrome, Retinitis Pigmentosa, Hydrocephalus, Hereditary Sensory and Autonomic Neuropathy Type IV, Mucopolysaccharidosis Type III, Corneal Dystrophies, Erythropoietic Protoporphyria, Fabry Disease, Werdnig-Hoffman Disease, Hypoposphatasia, Coats Disease, Fanconi Anemia, Niemann Pick Disease, Crigler-Najjar Syndrome, Hemophilia A, Hemophilia B, Leukodystrophy, Sandhoff Disease, Usher Syndrome, Wolman Disease, Dupuytren's Contracture, Wolfram Syndrome, X-Linked Myotubular Myopathy, Canavan Disease, Ehler's Danlos Syndrome, Epidermolysis Bullosa, Osteogenesis Imperfecta, Short Bowel Syndrome, Giant Axonal Neuropathy, Paroxysmal Nocturnal Hemoglobinuria, Phelan-McDermid Syndrome, Retinoschisis, Beta-Thalassemia, Hypophosphatasia, Propionic Acidemia, Cholesteryl Ester Storage Disease, Cystinosis, Glycogen Storage Disease Type II Pompe Disease, Mucopolysaccharidoses (MPS I H-S Hurler-Scheie), Mucopolysaccharidoses (Type II (Hunter syndrome)), and Mucopolysaccharidoses (Type IV (Morquio)).

The compositions of the disclosure may be used to treat a disease or disorder by use of a therapeutic transgene encoding for an exogenous nucleic acid sequence or exogenous amino acid sequence. For certain diseases or disorders the therapeutic transgene can include [Disease (therapeutic transge): Beta-Thalassemia (HBB T87Q, BCL11A shRNA, IGF2BP1), Sickle Cell Disease (HBB T87Q, BCL11A shRNA, IGF2BP1), Hemophilia A (Factor VIII), Hemophilia B (Factor IX), X-linked Severe Combined Immunodeficiency (Interleukin 2 receptor gamma (IL2RG)), Hypophosphatasia (Tissue Non-specific Alkaline Phosphatase (TNAP)), Osteopetrosis (TCIRG1), Glycogen Storage Disease Type II (Pompe Disease) (Alpha Glucosidase (GAA)), Alpha-Galactosidase A Deficiency (Fabry disease) (Alpha-galactosidase A (GLA)), Mucopolysaccharidosis Type I (MPS I) (Alpha-L-iduronidase (IDUA)), Mucopolysaccharidosis Type II (MPS II) (Iduronate 2-sulfatase (IDS)), Mucopolysaccharidosis Type IIIA (MPS IIIA) (sulfoglycosamine-sulfohydrolase (SGSH)), Mucopolysaccharidosis Type IIIB (MPS IIIB) (N-alpha-acetylglucosaminidase (NAGLU)), Mucopolysaccharidosis Type IV A (MPS IVA) (Morquio) (N-acetylgalactosamine-6-sulfate sulfatase (GALNS)), Mucopolysaccharidosis Type IV B (MPS IVB) Beta-galactosidase (GLB1 (Beta-galactosidase (GLB1)), Cholesteryl Ester Storage Disease (CESD) (Lysosomal acid lipase (LIPA)), Cystinosis (Cystinosin lysosomal cystine transporter (CTNS)), X-linked chronic granulomatous disease (X-CGD) (CYBB), Wiskott-Aldrich Syndrome (WAS) (WAS), X-linked Adrenoleukodystrophy (X-ALD) (ABCD1), Metachromatic leukopdystrophy (MLD) (ARSA), Phenylketonuria (PAH), Methylmalonic academia (MMUT), Propionic Acidemia (PCCA, PCCB), Retinitis Pigmentosa (RPE65), Usher Syndrome (MYO7A), and Gaucher Disease (GBA).

In preferred aspects, the treatment of a malignant disease or disorder comprises adoptive cell therapy. For example, in one aspect, the disclosure provides modified cells that express at least one disclosed antibody (e.g., scFv) and/or CAR comprising an antibody (e.g., scFv) that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or CAR expression level to ensure a minimal level of cell function and CAR expression. Modified cells can be formulated in a pharmaceutically acceptable carrier at a prescribed density with one or more reagents to inhibit further expansion and/or prevent cell death.

Any can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In one aspect, the administration is systemic. Systemic administration can be any means known in the art and described in detail herein. Preferably, systemic administration is by an intravenous injection or an intravenous infusion. In one aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising an antibody (e.g., scFv) or a CAR comprising an antibody (e.g., scFv) the antibody or CAR specifically binds to an antigen on a tumor cell. In aspects where the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34+ hematopoietic cells capable of giving rise to both mature myeloid and lymphoid cell types including T cells, NK cells and B cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, tissue, biopsy. The term also includes a cell that is removed from at least one, some or all components as the cell is found in non-naturally occurring environments, for example, culture, cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cells, substantially pure cells and cells cultured in a medium that is non-naturally occurring. Isolated cells may be obtained from separating the desired cells, or populations thereof, from other substances or cells in the environment, or from removing one or more other cell populations or subpopulations from the environment. As used herein, the term “purify” or the like refers to increase purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” in intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy.

As used herein, the term “engager” refers to a molecule, e.g. a fusion polypeptide, which is capable of forming a link between an immune cell, e.g. a T cell, a NK cell, a NKT cell, a B cell, a macrophage, a neutrophil, and a tumor cell; and activating the immune cell. Examples of engagers include, but are not limited to, bi-specific T cell engagers (BiTEs), bi-specific killer cell engagers (BiKEs), tri-specific killer cell engagers, or multi-specific killer cell engagers, or universal engagers compatible with multiple immune cell types.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g. a prodrug, that when activated, triggers apoptosis and/or cell death of a therapeutic cell. Examples of safety switch proteins, include, but are not limited to suicide genes such as caspase 9 (or caspase 3 or 7), thymidine kinase, cytosine deaminase, B-cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refer to proteins or peptides that are capable of achieving a biological and/or pharmaceutical effect on an organism. A pharmaceutically active protein has healing curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. Pharmaceutically active proteins include an entire protein or peptide or pharmaceutically active fragments thereof. It also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, the cellular signal transduction. Signal transduction refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes but not limited to i) antigen specificity as it related to a unique chimeric antigen receptor (CAR) or T cell receptor (TCR), ii) engager specificity as it related to monoclonal antibodies or bispecific engager, iii) targeting of transformed cell, iv) targeting of cancer stem cell, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that, as used herein, relates to the transfusion of autologous or allogenic lymphocytes, identified as T or B cells, genetically modified or not, that have been expanded ex vivo prior to said transfusion.

A “therapeutically sufficient amount”, as used herein, includes within its meaning a non-toxic but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a therapeutically sufficient amount is sufficient and/or effective to ameliorate, reduce, and/or improve at least one symptom associated with a disease or condition of the subject being treated.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level-successful removal, adding, or altering a cell function/characteristics via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA-class I deficient, or HLA-class II deficient, or both, refers to cells that either lack, or no longer maintain, or have reduced level of surface expression of a complete MHC complex comprising a HLA class I protein heterodimer and/or a HLA class II heterodimer, such that the diminished or reduced level is less than the level naturally detectable by other cells or by synthetic methods. HLA class I deficiency can be achieved by functional deletion of any region of the HLA class I locus (chromosome 6p21), or deletion or reducing the expression level of HLA class-I associated genes including, not being limited to, beta-2 microglobulin (B2M) gene, TAP 1 gene, TAP 2 gene and Tapasin. HLA class II deficiency can be achieved by functional deletion or reduction of HLA-II associated genes including, not being limited to, RFXANK, CIITA, RFX5 and RFXAP. It was unclear, prior to this invention, whether HLA complex deficient or altered iPSCs have the capacity to enter development, mature and generate functional differentiated cells while retaining modulated activity. In addition, it was unclear, prior to this invention, whether HLA complex deficient differentiated cells can be reprogrammed to iPSCs and maintained as pluripotent stem cells while having the HLA complex deficiency. Unanticipated failures during cellular reprogramming, maintenance of pluripotency and differentiation may related to aspects including, but not limited to, development stage specific gene expression or lack thereof, requirements for HLA complex presentation, protein shedding of introduced surface expressing modalities, need for proper and efficient clonal reprogramming, and need for reconfiguration of differentiation protocols.

### EXAMPLES

### Example 1-S44P Mutation in Cas-CLOVER Results in Increased In Vitro Gene Editing Activity Relative to Wildtype Cas-CLOVER

This example illustrates that Cas-CLOVER amino acid sequences harboring a S44P mutation in the Clo051 nuclease domain demonstrate increased in vitro gene editing activity in several cell lines compared to wild type Cas-CLOVER sequence.

### Gene Editing in Huh7 Cell Line

Huh7 cells were seeded at a density of 5E5 cells/ml in 12-well plates and transfected with a mix comprising a non-saturating amount 0.2 μg/ml of gRNA pairs targeting an intron of the albumin gene, the APOC3 gene or the TTR gene, 0.5 μg/ml of either wild type Cas-CLOVER mRNA (encoded by nucleic acid sequence of SEQ ID NO: 11) or a Cas-CLOVER mRNA comprising the S44P mutation (encoded by nucleic acid sequence of SEQ ID NO: 36), and 15 μl/ml of lipofectamine messengerMAX reagent (Thermo Fisher, Inc) in accordance with the manufacturer's instructions. After 48 hr, the medium was removed, the cells were lysed and genomic DNA was isolated from the cell extracts using a QuickExtract Kit (Lucigen Corp.) in accordance with the manufacturer's instructions.

The isolated genomic DNA was then analyzed using PCR by designing primers flanking the specific target sequence: Albumin-Fwd: AAGACGTGTGTGGGGATCAG (SEQ ID NO: 51) and Albumin-Rev: GAGCAAAGGCAATCAACACCC (SEQ ID NO: 52); APOC3-Fwd: CTCAGCCCTGCTCTTTCCTC (SEQ ID NO: 53) and APOC3-Rev: CTCGCAGGATGGATAGGCAG (SEQ ID NO: 54); TTR-Fwd: ATTGAACCCCAAGAACCACAT (SEQ ID NO: 55) and TTR-Rev: CTGCCTCCTAGATTCAAGGG (SEQ ID NO: 56). PCR products were isolated, and the DNA sequence was determined by direct Sanger sequencing. Indel rates for the wild type and S44P Cas-CLOVER gene editing rates were quantified using ICE analysis (Synthego Corp.). The indel percentages for wild type and S44P in Huh7 cell line is shown in Table 2.

TABLE 2
Indel Percentages in Huh7 Cell Line

| | ALBUMIN | APOC3 | TTR |
|---|---|---|---|
| Huh7 WT Cas-CLOVER | 48 | 42 | 34 |
| Huh7 S44P Cas-CLOVER | 60 | 34 | 22 |

As shown in Table 2, the in vitro activity of the S44P mutant is similar to that of the wild type Cas-CLOVER sequence in these cell lines.

### Gene Editing in HepG2 Cell Line

A mix comprising a non-saturating amount 0.2 μg/ml of gRNA pairs targeting an intron of the albumin gene, the APOC3 gene or TTR gene, 0.5 μg/ml of either wild type Cas-CLOVER mRNA (encoded by nucleic acid sequence of SEQ ID NO: 11) or a Cas-CLOVER mRNA comprising the S44P mutation (encoded by nucleic acid sequence of SEQ ID NO: 36) was electroporated into 5E5 HepG2 cells using Lonza Nucleofector in accordance with the manufacturer's instructions. Electroporated cells were seeded at a density of 5E6 cells/ml and incubated in medium for 48 hrs. After 48 hr, the medium was removed, the cells were lysed and genomic DNA was isolated from the cell extracts using a QuickExtract Kit (Lucigen Corp.) in accordance with the manufacturer's instructions.

The isolated genomic DNA was then analyzed using PCR by designing primers flanking the specific gene target sequence: Albumin-Fwd: AAGACGTGTGTGGGGATCAG (SEQ ID NO: 51) and Albumin-Rev: GAGCAAAGGCAATCAACACCC (SEQ ID NO: 52); APOC3-Fwd: CTCAGCCCTGCTCTTTCCTC (SEQ ID NO: 53) and APOC3-Rev: CTCGCAGGATGGATAGGCAG (SEQ ID NO: 54); TTR-Fwd: ATTGAACCCCAAGAACCACAT (SEQ ID NO: 55) and TTR-Rev: CTGCCTCCTAGATTCAAGGG (SEQ ID NO: 56). PCR products were isolated and the DNA sequence determined by direct Sanger sequencing. Indel rates for the wild type and S44P Cas-CLOVER gene editing rates were quantified using ICE analysis (Synthego Corp.). The percentage of indels for wild type and S44P Cas-CLOVER variant in HepG2 cell line is shown in Table 3.

TABLE 3
Indel Percentages in HepG2 Cell Line

| | ALBUMIN | APOC3 | TTR |
|---|---|---|---|
| HepG2 WT Cas-CLOVER | 40 | 57 | 12 |
| HepG2 S44P Cas-CLOVER | 39 | 82 | 22 |

As shown in Table 3, the S44P mutation results in an approximately 1.4-fold to 1.8-fold improvement in indel rates at the APOC3 and TTR loci compared to wild type Cas-CLOVER sequence.

### Gene Editing in HPSCs

A mix comprising a non-saturating amount 0.2 μg/ml of gRNA pairs targeting HBG1 gene (SEQ ID NO: 67 and SEQ ID NO: 68), and 1.5 or 4.0 μg/ml of either wild type Cas-CLOVER mRNA (encoded by nucleic acid sequence of SEQ ID NO: 11) or a Cas-CLOVER mRNA comprising the S44P mutation (encoded by nucleic acid sequence of SEQ ID NO: 36) was electroporated into 5E5 HSPC cells using Lonza Nucleofector in accordance with the manufacturer's instructions. Electroporated cells were seeded at a density of 5E6 cells/ml and incubated in DMEM with 10% FBS medium for 48 hrs. After 48 hr, the medium was removed, the cells were lysed and genomic DNA was isolated from the cell extracts using a QuickExtract Kit (Lucigen Corp.) in accordance with the manufacturer's instructions.

For HSPC cells, gene editing at the HBG locus was measured using Next Generation Sequencing (“NGS”). Briefly, genomic DNA samples were subjected to PCR amplification using DNA primers flanking the HBG1 gene exon 1 (shown in underlined font) that further contain Illumina partial adapters: Fwd ACACTCTTTCCCTACACGACGCTCTTCCGATCTGCAGTATCCTCTTGGGGG (SEQ ID NO: 57); and Rev GACTGGAGTTCAGACGTGTGCTCTTCCGATCTACCTCAGACGTTCCAGAAGC (SEQ ID NO: 58). The resulting PCR amplicons underwent a second PCR reaction using primers containing Illumina P5 and P7 sequences (Illumina Corp) and a unique index sequence (New England Biolabs). The final amplicons were pooled at equimolar concentrations, and analyzed using a Miseq benchtop sequencer following standard procedures for Amplicon-seq according to the manufacturer (Illumina Corp). Sequence data were analyzed using a CRISPResso2 program to determine the frequency of insertions/deletions in each sample. The results are shown in Table 4.

TABLE 4
Frequency of insertions and deletions

| Concentration | | |
|---|---|---|
| (μg/ml) | Wild Type Cas-CLOVER | S44P Cas-CLOVER |
| 1.5 | 18 | 22 |
| 4.0 | 37 | 49 |

As shown in Table 4, the S44P mutation results in a modest improvement in indel rates at each concentration at the HBG1 loci in HPSCs compared to wild type Cas-CLOVER sequence.

### Example 2-S44P Mutation in Cas-CLOVER Results in Increased In Vivo Gene Editing Activity Relative to Wildtype Cas-CLOVER

This example illustrates that Cas-CLOVER amino acid sequences harboring a S44P mutation in the Clo051 nuclease domain demonstrate increased in vivo gene editing activity at multiple genomic loci compared to wild type Cas-CLOVER.

### Psck9 Gene Editing

### A. LNP Preparation

To formulate the lipid nanoparticles (LNPs), various percentages of the terpene lipidoid HMA-404, the phospholipid DOPC, the structural lipid cholesterol (Chol) and 1,2-dimyristoyl-sn-glycerol methoxypolyethylene glycol (DMG-PEG2000; Avanti Polar Lipids, Alabaster, Alabama, USA) were combined to prepare LNP compositions. RNA molecules were encapsulated in lipid nanoparticles comprising about 40.75% of HMA-404 by moles, about 51.75% of cholesterol by moles, about 5% of DOPC by moles, and about 2.5% of DMG-PEG2000 by moles. The ratio of lipid to nucleic acid in the nanoparticles was about 120:1 (weight/weight) and the total lipid of 25 mM.

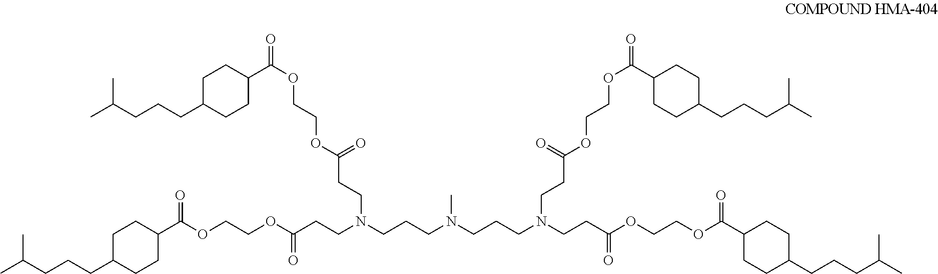 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Compound HMA-404 was prepared as described in PCT Application No. PCT/US2023/061005. The crude was purified by silica gel flash chromatography (eluent: 5% MeOH/DCM). 1H NMR (400 MHz, Chloroform-d) δ 4.38-4.12 (m, 16H), 2.90-2.78 (m, 4H), 2.71 (t, J=6.8 Hz, 8H), 2.61 (s, 3H), 2.57-2.36 (m, 12H), 2.35-2.04 (m, 4H), 2.01-1.62 (m, 17H), 1.60-0.96 (m, 45H), 0.94-0.67 (m, 30H).

The mRNA molecules were further capped using CleanCap® (TriLink Corp), and all cytidine residues in the mRNA were substituted with 5-methylcytidine (5-MeC).

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temp and then placed on a hot plate maintained at a temperature range of 50-55° C. Subsequently, the hot lipid stock solutions were combined to yield desired final mol percentages.

A 1 mg/ml solution of a pair of gRNAs targeting the first exon of the mouse pcsk9 gene (SEQ ID NO: 59 and SEQ ID NO: 60) and either mRNA encoding 5′-CleanCap-5MeC-Cas-CLOVER (SEQ ID NO: 69) or 5′-CleanCap-5MeC-Cas-CLOVER S44P (SEQ ID NO: 70) and to be incorporated into the LNPs were individually added to 150 mM sodium acetate buffer (pH 5.2) to form a stock solution and kept on ice. The lipid phase was mixed with the aqueous mRNA phase inside a microfluidic chip using a NanoAssemblr® instrument (Precision Nanosystems, Vancouver, BC, Canada) according to the manufacturer's instructions to form LNP compositions comprising encapsulated mRNAs. Nanoassemblr process parameters for mRNA encapsulation are shown in the Table 5.

TABLE 5
Nanoassemblr process parameters

| Total flow rate (ml/min) | Lipid phase:aqueous (RNA) phase (v/v) |
|---|---|
| 20 | 1:3 |

The resultant mRNA LNP compositions were then transferred to a Repligen Float-A-Lyzer dialysis device-having a molecular weight cut off (MWCO) of 8-10 kDa (Spectrum Chemical Mfg. Corp, CA, USA) and processed by dialysis against phosphate buffered saline (PBS) (dialysate:dialysis buffer volume at least 1:200 v/v), pH 7.4 overnight at 4° C. (or alternatively room temperature for at least 4 hours), to remove the 25% ethanol and achieve a complete buffer exchange. In some experiments the LNPs were further concentrated in an Amicon® Ultra-4 centrifugal filter unit, MWCO-30 kDa (Millipore Sigma, USA) spun at ˜4100×g in an ultracentrifuge. The mRNA LNPs were then stored at 4° C. until further use. The average particle size diameter of the LNPs was approximately 70 nm.

### B. Pilot Study

Adult female BALB/C mice (n=2/group) were intravenously co-administered a pair of psck9-targeting gRNAs (SEQ ID NO: 59 and SEQ ID NO: 60), and either mRNA encoding 5′-CleanCap-5MeC-Cas-CLOVER (SEQ ID NO: 69) or 5′-CleanCap-5MeC-Cas-CLOVER S44P (SEQ ID NO: 70) at 1.0 mg/kg formulated in the LNP compositions in Example 2, Section A. One group of mice was treated with vehicle (PBS, Thermo Fisher Scientific, USA) as a negative control.

After seven days post-administration, the mice were euthanized, and DNA was isolated from the liver of treated and untreated mice. Briefly, the livers were resected after euthanasia, flash frozen in liquid nitrogen, mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissues were incubated at 56° C. for 30 minutes, and column-purified using a Monarch Genomic DNA Purification kit from New England Biolabs according to the manufacturer's instructions. Final DNA elution was performed in 50 μL of elution buffer (10 mM Tris-Cl, pH 8.5). The concentration and purity of DNA samples was assessed by measuring absorbance at 260 and 280 nm.

The extent of gene editing observed for Cas-CLOVER and Cas-CLOVER S44P mRNA delivered to the mice was measured by Droplet Digital PCR (ddPCR) using Drop-off assays that contain a fluorescent probe that hybridizes with Cas-CLOVER target site. The percentage of indels for wild type Cas-CLOVER and Cas-CLOVER S44P at the psck9 locus in the liver of treated mice are shown in Table 6.

TABLE 6
Percentage of indels at the Pcsk9 locus in mouse liver

| Concentration | PBS | | |
|---|---|---|---|
| (mg/kg) | Control | Cas-CLOVER WT | Cas-CLOVER S44P |
| 1.0 | 0 | 15 | 29 |

As shown in Table 6, Cas-CLOVER harboring a S44P mutation resulting in increased indel percentages about 1.9-fold compared to wild type Cas-CLOVER demonstrating the improved in vivo activity of the S44P mutant.

### C. Follow Up Study

Adult female BALB/C mice (n=2/group) were intravenously co-administered a pair of psck9-targeting gRNAs (SEQ ID NO: 59 and SEQ ID NO: 60), and either mRNA encoding 5′-CleanCap-5MeC-Cas-CLOVER (SEQ ID NO: 69) or 5′-CleanCap-5MeC-Cas-CLOVER S44P (SEQ ID NO: 70) at 0.75 mg/kg or 1.5 mg/kg formulated in the LNP compositions in Example 2A. One group of mice was treated with vehicle (PBS, Thermo Fisher Scientific, USA) as a negative control.

After seven days post-administration, the mice were euthanized and DNA was isolated from five tissue types from the mice in each group: liver, heart, spleen, lung, and kidney. Briefly, liver biopsies were resected after euthanasia, flash frozen in liquid nitrogen, mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissues were incubated at 56° C. for 30 minutes, and column-purified using a Monarch Genomic DNA Purification kit from New England Biolabs according to the manufacturer's instructions. Final DNA elution was performed in 50 μL of elution buffer (10 mM Tris-Cl, pH 8.5). The concentration and purity of DNA samples was assessed by measuring absorbance at 260 and 280 nm.

The extent of gene editing observed for Cas-CLOVER and Cas-CLOVER S44P mRNA delivered to the mice was measured by Droplet Digital PCR (ddPCR) using Drop-off assays that contain a fluorescent probe that hybridizes with Cas-CLOVER target site. The percentage of indels for wild type Cas-CLOVER and Cas-CLOVER S44P at the psck9 locus in the liver of treated mice are shown in Table 7.

TABLE 7
Percentage of indels at pcsk9 locus of mouse liver

| Concentration | PBS | | |
|---|---|---|---|
| (mg/kg) | Control | Cas-CLOVER WT | Cas-CLOVER S44P |
| 0.75 | 0 | 0.2 | 8.0 |
| 1.5 | 0 | 11.4 | 33.8 |

As shown in Table 7, Cas-CLOVER harboring a S44P mutation resulting in increased indel percentages between about 3-40-fold compared to wild type Cas-CLOVER demonstrating the improved in vivo activity of the S44P mutant.

1. A polypeptide comprising the amino acid sequence of SEQ ID NO: 35.

2. A polynucleotide encoding the polypeptide of claim 1.

3. A vector comprising the polynucleotide of claim 2.

4. A pharmaceutical composition comprising the vector of claim 3 and at least one pharmaceutically acceptable excipient or diluent.

5. A method of modifying a target sequence in the genome of a plurality of cells comprising introducing to a population of unmodified cells a composition comprising:
a) a polypeptide comprising the amino acid sequence of SEQ ID NO: 35, or a polynucleotide encoding the polypeptide of SEQ ID NO: 35; and
b) at least one guide RNA (gRNA),
thereby generating a modification at the target sequence in the genome, and
wherein 1.1-fold to 50-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10.

6. The method of claim 5, wherein 1.2-fold to 1.8-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10.

7. The method of claim 5, wherein 3-fold to 40-fold of the plurality of cells comprise the modification at the target sequence in the genome in comparison to a plurality of modified cells introduced with a composition comprising a polypeptide comprising the amino acid sequence of SEQ ID NO: 10 or a polynucleotide encoding the polypeptide of SEQ ID NO: 10.

8. The method of claim 5, wherein the modification at the target sequence in the genome is a deletion, a insertion, a substitution, a inversion and/or a relocation.

9. The method of claim 5, wherein the composition is encapsulated in at least one lipid nanoparticle comprising:
about 40.75% of a compound of HMA-404 by moles,
about 51.75% of cholesterol by moles,
about 5% of DOPC by moles, and
about 2.5% of DMG-PEG2000 by moles;
wherein HMA-404 is shown in the following structure: 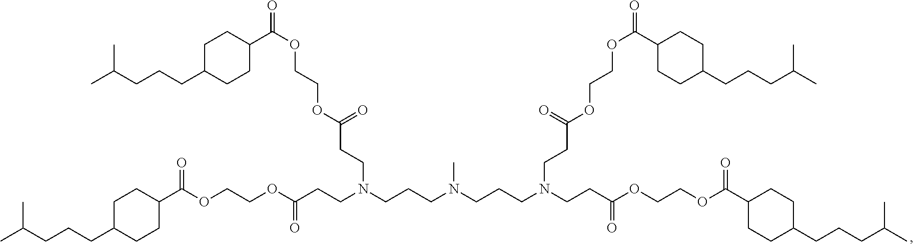 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]
wherein a polynucleotide encoding the polypeptide of SEQ ID NO: 35 is an RNA molecule, and wherein the ratio of lipid to RNA molecule in the at least one nanoparticle is about 120:1 (w/w).

10. The method of claim 5, wherein the plurality of cells comprise:
(a) a liver cell, preferably wherein the liver cell is a hepatocyte, a hepatic stellate cell, Kupffer cell or liver sinusoidal endothelial cell;
(b) a T-cell, preferably wherein the T-cell is an activated T-cell, a resting T-cell or a stem memory T cell (T_{SCM} cell); or
(c) a hematopoietic stem cell (HSC).

11. A cell modified according to the method of claim 5.

12. A composition comprising a population of cells modified according to the method of claim 5.

13. A method of treating at least one disease or disorder in a subject in need thereof comprising administering to the subject at least one therapeutically effective amount of the pharmaceutical composition of claim 4.

14. The method of claim 13, wherein the at least one disease or disorder is a liver disease or disorder, preferably wherein the liver disease or disorder is:
(a) a metabolic liver disorder;
(b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof.

15. The method of claim 13, wherein the at least one disease or disorder is cancer.

16. The method of claim 13, wherein the at least one disease or disorder is hemophilia A.

17. A method of treating at least one disease or disorder in a subject in need thereof comprising administering to the subject at least one therapeutically effective amount of the cell of claim 11.

18. The method of claim 17, wherein the at least one disease or disorder is cancer, hemophilia A, or a liver disease or disorder, preferably wherein the liver disease or disorder is:
(a) a metabolic liver disorder;
(b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof.

19. A method of treating at least one disease or disorder in a subject in need thereof comprising administering to the subject at least one therapeutically effective amount of the composition of claim 12.

20. The method of claim 19, wherein the at least one disease or disorder is cancer, hemophilia A, or a liver disease or disorder, preferably wherein the liver disease or disorder is:
(a) a metabolic liver disorder;
(b) a urea cycle disorder (UCD), preferably wherein the UCD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) or any combination thereof.
